SEQUENCE LISTING (27 sequences)

SEQ ID NO: 1 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 2 - RNA, 570 nt, Homo sapiens
  source 1..570: mol_type = mRNA
atgctggggagcagagctgtaatgctgctgttgctgctgccctggacagctcagggcagagctgtgcctgggggcagcagccctgcctggactcagtgccagcagctttcacagaagctctgcacactggcctggagtgcacatccactagtgggacacatggatctaagagaagagggagatgaagagactacaaatgatgttccccatatccagtgtggagatggctgtgacccccaaggactcagggacaacagtcagttctgcttgcaaaggatccaccagggtctgattttttatgagaagctgctaggatcggatattttcacaggggagccttctctgctccctgatagccctgtgggccagcttcatgcctccctactgggcctcagccaactcctgcagcctgagggtcaccactgggagactcagcagattccaagcctcagtcccagccagccatggcagcgtctccttctccgcttcaaaatccttcgcagcctccaggcctttgtggctgtagccgcccgggtctttgcccatggagcagcaaccctgagtccctaa

SEQ ID NO: 3 - DNA, 6252 bp, Homo sapiens
  source 1..6252: mol_type = genomic DNA
ttacggggcgccggtgggacaaacgcagccagacgccggacttgttgggtggcgttagaaagtaactctgtttttagtcttcttgaacaaaattacgtctaggtttgaaaaccaaacttttatgctttctataactttaggaactctaaaataagggacacacaaggcaaagcactaggagtcgctgttgggtattcccagcggggcttagtctacctcctcttcctgcggtgccctagtgagccactccgccacctctgaccctgttgcccccaaaactcgctggcatttcctattatttccagatatgggagagaaaaaaaaggaagcgggagccggactcctgacacagtccaagtggctgcatcttctacaggcatccatctgaaactcccatccgtgattgttccctccccagagaccccggtaacattcccgggtaacaagatgcccctggttatcaaattcccctagctcttgaggctggctggacgttatccctcagagggggatgagcatggcaaattgggacttgttattctgaaggattcgtgggtcctgtgaactctaattactttgaaatgggtctaggttgtgagatgtctcagagcactttagctcagctgttattactgtttctaaaggccacataaagggactctgatgggagacattcctcatggaggattcaattctataacatttctctcaataaaggctggtaaatagaccttcattaaaggaaccaagaatttaaatttctaggactcagaggggtggggtcctatacccagtcagagatcctacctagagcctagaccaagagaaaaacacagatggtctctcaaactgatttgatctgacttcgcaggtcattagatatagaatctccgaaaaaggtggatgctgagagacatagacagttcctacactttaagaaatctccatcttgaggtctcaaattgagaaagacttaacagacccatgagagttacagatccctaataacctgggctaaataatccatgtctgccgggcgcagtggctcacgcctgtaatcccagcactttgggaggccgaggtgggcggatcacctgaggtcgggagttcgagaccaacctgaccaatatggagaaaccccgtctccactaaaaatacaaaattagccgggcctggtgacgggagcctgtaatcccagctacttgggaggctgaggcaggagaatcatttgaacctgggaggcggaggttgcagtgagccgagatggcaccattgcactccagcctggtcagtaagagcgaaactccgtctcaaaaaaaagaaaaaaaaaaagaaaagaaaaaaggatactgtgaggagacacaagagcatccatgacatagattatttagctcagctgtaattactgtttctaatacagtaatattagatggtgatctgcctgcctcggcctcccaaagttctgggattacaggtgtgagccaccgcgcccagcctttttttttttttttttttgagacaggatctcactctgttgcccatgcttaagcgcattggccctctcactcactgtagcctcaacctcctgggctcaagcgatcctcccacttcagcctcccaactagctgtaactacaggcactggccaccaaacccagataattttttttttcctgtagaggtggggttttgccacgttacccaggctggtcttgaactcttaagctcaagcgatcctcctgcctcggccccccaaagttctgggattacaggcatgagccaccatacctggcgtacagtatccagtgtaatgcagtgattaaaaattcaggatccagaccgggatggtggcttgtgcctgtagtcccaggggtggaggttgcagtgaacggaaatggtgccactgcattccagcctgggtgacagagtgagaccctgtctcaacaaaaccccccaaaaaccaagaacaaaaaagaatgcaggatctgatgctagattgtctgcattagaactctagccacttaagctgggtgtggtggctcatgcctgtaatcccagcacttttggaggccgcaggcgggtggatcacctgagattgggagttcaagaccagcctgaccaacatggagaaaccctgtttctactaaaaatacaaaattagccaccgggcatggtggtgcatgcctgtaatcccagctacttgggaggctgaggcaggagaatcacttgaacccgggaggcagaggttacggtgagatgagatggcaccattgcactccagcctgggcaacaagagcgaaactccatctcaaaaaaaaaaaaaaaaaaaaaggaattctagccacttagaagctctgtgatcttgggcaaattgcttatctttgcacctcagcctcctcctctgtaatatagggtaatagtatctaccttaaagggttgttgtgaaaattaaatagtttagtacatgtaaagtgcttagacaaagtatttggcattaagcgagagttggatatattagccatcattattaaccacctgggggaacttcaactgatttggagtctaggcatacaactggaaagacctgcctaggagtgtcttgtgaatgcgatttgcataacggtttaggcccagctgacgtcaagggctccttatagctccaggtcagttgtagccctggatgtagttcctgccacgcaacagtcccacaatctccccaccaacccttcttcctacccaactcctgcagcaccaggaagtgaaacaaagaggcagagccctgtgcctccaactcacccttgtccctctcatcccatcccccaggctctacttcctcctccttttcatctttctttcatctcttatcttttagggctcccagaatggggaccagagatgggaagaacataggagacgttgtacacaagtaaggtgaactccctatcctgccccctcccctttccttattccattggtgtccaccttattagggagagaggcaaaacagttctcacccaaactcagataattctctgatgctggaaatgtttaatctaaagggtagatttccatttttttttttttttttttgagacagagtcttgctctgtcacccaggctggagtgcagtggcgccatctcggctcactgcaacctctgcctcctggcttcaagcgattctcttgcctcagcctcccgagtagctgggactataggcgcccaccaccgtgctggctcatttttgtatttttagtagagacaggatttcaccatgatggccaggctggtctcgaactcctgacctcatgatccgcctgcctcggcctcccaaagtactgggattacaggcgtgagccactgtacccggcccttggtagatttaacttagaatcgtaatatttttttttcttctcttagctcatacctacagaatcataatatttgaaccagaagtgtcattgggcagttttgaatagctctaagggaagggagacctccattcaggacaagtttctcagaagaaaagggtcaacctcttgggggaggctttgggagccagctgtgtggtcaccgatggcctcattctgacgtcttcgaaattgttctgggaccctccactggggtcggggcagtcccggctttggaccaccttccactcccacgcccaacctcacactcttagctgtttcactcgatgttgcatcatggagggtgatgaaatcggtgtcagtggattttacccatggatgcaacaagctgaaggaccagccagagtcattgacagtgcaccttcgactacccagaactcctgggcttcctagccatggggtccaaagctgggactgccccgaccccagtggagggtcccagaacaatttggatgacgtcagaatgaggccatgggactaggtgctggaatgtctaagttgaacttccaggccttatttgcactagtcctgaaaaaaacatcatccaactcttatagagcctatgaaatcttgggccactagggttgaggagtcaggtggttcttagtcaataaccctcttcccacaagagcctttctaacctccactgtgaggcctgaaatggggagcaataagacctcatactggcttcccagttctccaagttccttcatgcgcattctctcccatgaaaccaggaccatccagttgaaataatgttgtttccaactgagaaaaagaagcccgtttattcctaatagggggcatcaggtaggaatcaaacttcattgcaaacagctcaccatcctattgggagatgaatggatgtttctctgttttgctttttcctcaagcaggaggaagtgaggaaattaggtttggggtggggtaggggtatagctttgagaggcaaaaagatcagggaaagatcaaacaggaaggaacttgagaccagattaatttaaatatttgttctcccttacccctcccaccccatccccgctgtgccccccatccccgccccttctatagctatttcgattcctggagagcattacacatgtgtcccatcccaggcctctagccacagcaaccacactactcatttcccctggaactgaggctgcatacctgggctccccacagagggggatgatgcagggaggggaatcccacctgctgtgagtcacctgctggtataaagggcgggccttacaatgcagggaccttaaaagactcagagacaaagggagaaaaacaacaggaagcagcttacaaactcggtgaacaactgagggaaccaaaccagagacgcgctgaacagagagaatcaggctcaaagcaagtggaagtgggcagagattccaccaggactggtgcaaggcgcagagccagccagatttgagaagaaggcaaaaagatgctggggagcagagctgtaatgctgctgttgctgctgccctggacagctcagggcagagctgtgcctgggggcagcagccctgcctggactcagtgccagcagctttcacagaagctctgcacactggcctggagtgcacatccactagtgggacacatggtgagtggcagcccctggagcctaacaggagtccaggctctccaaggctgtggcagaagaccgtgaccttgagtggaagctggagggttgaaggccattagggagtaagagaggacaagagagtagggttcctgggagagtcatgggcctgagggtccaggttggcttcagaagtactatcttacttcttcattctttccacctcttccttcattccaggatctaagagaagagggagatgaagagactacaaatgatgttccccatatccagtgtggagatggctgtgacccccaaggactcagggacaacagtcaggtaccactgggatgtggctgggcaatgaaggagaggggactgagaacatggctgggtaccatggtaaaccagaagttgtgtctgaaaatagtaagaaactgggtgagtcttcagtgaatggagtaggaagagggtgtcctctttcattgctttcttttctccctagttctgcttgcaaaggatccaccagggtctgattttttatgagaagctgctaggatcggatattttcacaggggagccttctctgctccctgatagccctgtgggccagcttcatgcctccctactgggcctcagccaactcctgcaggtatgaagtaggggcgtggaggatgggggcttgcaggtgtcagagacagagggttgggggttaagggtttagagtcttctctgactgtgtcctatgtcctttcagcctgagggtcaccactgggagactcagcagattccaagcctcagtcccagccagccatggcagcgtctccttctccgcttcaaaatccttcgcagcctccaggcctttgtggctgtagccgcccgggtctttgcccatggagcagcaaccctgagtccctaaaggcagcagctcaaggatggcactcagatctccatggcccagcaaggccaagataaatctaccaccccaggcacctgtgagccaacaggttaattagtccattaattttagtgggacctgcatatgttgaaaattaccaatactgactgacatgtgatgctgacctatgataaggttgagtatttattagatgggaagggaaatttggggattatttatcctcctggggacagtttggggaggattatttattgtatttatattgaattatgtacttttttcaataaagtcttatttttgtggcta

SEQ ID NO: 4 - DNA, 20 bp, synthetic construct
  misc_feature 1..20: note = IL23p19 Forward primer
  source 1..20: mol_type = other DNA
gcagattccaagcctcagtc

SEQ ID NO: 5 - DNA, 20 bp, synthetic construct
  misc_feature 1..20: note = IL23p19 Reverse primer
  source 1..20: mol_type = other DNA
ttcaacatatgcaggtccca

SEQ ID NO: 6 - DNA, 19 bp, synthetic construct
  misc_feature 1..19: note = GAPDH Forward primer
  source 1..19: mol_type = other DNA
acagtccatgccatcactg

SEQ ID NO: 7 - DNA, 19 bp, synthetic construct
  misc_feature 1..19: note = GAPDH Reverse primer
  source 1..19: mol_type = other DNA
agtagaggcagggatgatg

SEQ ID NO: 8 - DNA, 20 bp, synthetic construct
  misc_feature 1..20: note = human IL-17 Forward primer
  source 1..20: mol_type = other DNA
caaccgatccacctcacctt

SEQ ID NO: 9 - DNA, 19 bp, synthetic construct
  misc_feature 1..19: note = human IL-17 Reverse primer
  source 1..19: mol_type = other DNA
ggcactttgcctcccagat

SEQ ID NO: 10 - DNA, 21 bp, synthetic construct
  misc_feature 1..21: note = IL-1B forward primer
  source 1..21: mol_type = other DNA
acagatgaagtgctccttcca

SEQ ID NO: 11 - DNA, 21 bp, synthetic construct
  misc_feature 1..21: note = IL-1B reverse primer
  source 1..21: mol_type = other DNA
gtcggagattcgtagctggat

SEQ ID NO: 12 - DNA, 21 bp, synthetic construct
  misc_feature 1..21: note = LIF forward primer
  source 1..21: mol_type = other DNA
cagtgccaatgccctctttat

SEQ ID NO: 13 - DNA, 20 bp, synthetic construct
  misc_feature 1..20: note = LIF reverse primer
  source 1..20: mol_type = other DNA
ggccacatagcttgtccagg

SEQ ID NO: 14 - DNA, 21 bp, synthetic construct
  misc_feature 1..21: note = TNF-alpha forward primer
  source 1..21: mol_type = other DNA
ggctccaggcggtgcttgttc

SEQ ID NO: 15 - DNA, 20 bp, synthetic construct
  misc_feature 1..20: note = TNF-alpha reverse primer
  source 1..20: mol_type = other DNA
agacggcgatgcggctgatg

SEQ ID NO: 16 - DNA, 22 bp, synthetic construct
  misc_feature 1..22: note = COX-2 forward primer
  source 1..22: mol_type = other DNA
gaatcattcaccaggcaaattg

SEQ ID NO: 17 - DNA, 20 bp, synthetic construct
  misc_feature 1..20: note = COX-2 reverse primer
  source 1..20: mol_type = other DNA
tctgtactgcgggtggaaca

SEQ ID NO: 18 - DNA, 21 bp, synthetic construct
  misc_feature 1..21: note = MCP-1 (CCL2) forward primer
  source 1..21: mol_type = other DNA
tcatagcagccaccttcattc

SEQ ID NO: 19 - DNA, 20 bp, synthetic construct
  misc_feature 1..20: note = MCP-1 (CCL2) reverse primer
  source 1..20: mol_type = other DNA
tagcgcagattcttgggttg

SEQ ID NO: 20 - DNA, 25 bp, synthetic construct
  misc_feature 1..25: note = MIP-1alpha (CCL3) forward primer
  source 1..25: mol_type = other DNA
ttgtgattgtttgctctgagagttc

SEQ ID NO: 21 - DNA, 20 bp, synthetic construct
  misc_feature 1..20: note = MIP-1alpha (CCL3) reverse primer
  source 1..20: mol_type = other DNA
cggtcgtcaccagacacact

SEQ ID NO: 22 - DNA, 21 bp, synthetic construct
  misc_feature 1..21: note = MIP-3alpha (CCL20) forward primer
  source 1..21: mol_type = other DNA
ttcacccaagtctgttttgga

SEQ ID NO: 23 - DNA, 18 bp, synthetic construct
  misc_feature 1..18: note = MIP-3alpha (CCL20) reverse primer
  source 1..18: mol_type = other DNA
gaaggctgtgacaatgct

SEQ ID NO: 24 - DNA, 18 bp, synthetic construct
  misc_feature 1..18: note = CXCL1 forward primer
  source 1..18: mol_type = other DNA
agtggcactgctgctcct

SEQ ID NO: 25 - DNA, 20 bp, synthetic construct
  misc_feature 1..20: note = CXCL1 reverse primer
  source 1..20: mol_type = other DNA
tggatgttcttggggtgaat

SEQ ID NO: 26 - DNA, 18 bp, synthetic construct
  misc_feature 1..18: note = CXCL2 forward primer
  source 1..18: mol_type = other DNA
ctgctcctgctcctggtg

SEQ ID NO: 27 - DNA, 18 bp, synthetic construct
  misc_feature 1..18: note = CXCL2 reverse primer
  source 1..18: mol_type = other DNA
agggtctgcaagcactgg

(19) United States
(12) Patent Application Publication
(10) Pub. No.: US 2024/0335395 A1
(43) Pub. Date: Oct. 10, 2024

(54) COMPOSITIONS AND METHODS FOR PREVENTING AND/OR TREATING DISEASE ASSOCIATED WITH IL-23 EXPRESSION

(71) Applicant: ALLSPIM, Paris (FR)

(72) Inventors: Jean-Marie ANDRIEU, Paris (FR); Louis LU, Paris (FR)

(73) Assignee: ALLSPIM, Paris (FR)

(21) Appl. No.: 18/578,957

(22) PCT Filed: Jul. 15, 2022
(86) PCT No.: PCT/EP2022/069944

§ 371 Date: Jan. 12, 2024

(30) Foreign Application Priority Data
Jul. 15, 2021 (EP) 21305991.8

(51) Int. Cl. A61K 31/12; A61P 15/08; A61P 17/06; A61P 19/02; A61P 19/06; A61P 21/00; A61P 27/02; A61P 29/00; A61P 37/06

(52) U.S. Cl. CPC A61K 31/12; A61P 15/08; A61P 17/06; A61P 19/02; A61P 19/06; A61P 21/00; A61P 27/02; A61P 29/00; A61P 37/06

(57) ABSTRACT

The present invention relates to compositions comprising 2′,4′-dihydroxy-3′,6′-dimethoxychalcone or a pharmaceutically acceptable salt thereof for use as a medicament. In particular, the present invention relates to compositions comprising 2′,4′-dihydroxy-3′,6′-dimethoxychalcone or a pharmaceutically acceptable salt thereof for use in the prevention and/or treatment of a disease associated with IL-23/IL-17 axis activation, except cancers.

The present invention relates to compositions comprising 2′,4′-dihydroxy-3′,6′-dimethoxychalcone or a pharmaceutically acceptable salt thereof for use as a medicament. These compositions are particularly useful in the prevention and/or treatment of a disease associated with IL-23 expression in humans.

### BACKGROUND OF THE INVENTION

The IL-23 cytokine is a member of the IL-12 family of cytokines with the biologically active heterodimer being composed of a p40 subunit, which it shares with IL-12, and a p19 subunit. IL-23 is mainly secreted by activated macrophages and dendritic cells (DCs) in peripheral tissues, and is implicated in the development, maintenance and expansion of several types of immune cells.

For example, IL-23 induces the proliferation of Th17 cells, differentiated from naïve CD4⁺ T-cell precursors, via binding to the IL-23 receptor (IL-23R; Aggarwal et al., 2003). The Th17 pathway plays a protective role against extracellular bacterial, mycobacterial and fungal infections, such as Candida albicans, Klebsiella pneumoniae, Listeria monocytogenes and Mycobacterium tuberculosis (see, for example, Hernández-Santos and Gaffen, 2012; Happel et al., 2005; Meeks et al., 2009). In particular, Th17 cells induce local tissue inflammation via production of cytokines such as IL-17 (specifically IL-17A and IL-17F), IL-22, and granulocyte-macrophage colony-stimulating factor (GM-CSF). IL-17A in turn binds to cognate receptors to activate signal transduction pathways mediated by NF-kB, AP-1, and C/EBP, driving the expression of a variety of molecules including pro-inflammatory cytokines, such as IL-1β, IL-6, IL-8, and TNF-α, chemokines, metalloproteinases, and inflammatory mediators (Fragoulis et al., 2016).

In addition to differentiated Th17 cells of the adaptive immune system, many innate immune cells located in non-lymphoid tissues are important in resisting infection and are also stimulated by IL-23. These cells, termed “type-17” cells, include subsets of γδT cells, natural killer T (NKT) cells, and innate lymphoid cells (ILCs), among others, and ubiquitously express the transcription factor retinoic-acid-receptor-related orphan receptor-γ (RORγt) and IL-23R. Thus, IL-23 represents a central cytokine straddling the innate and adaptive branches of the immune system which promotes early local immune responses.

A deregulation of IL-23 and of IL-23-controlled downstream pathways, also, via the continued and/or inappropriate expression of IL-23 is central in multiple auto-immune and inflammatory conditions and more generally of all situations comprising an inflammatory process. A plethora of studies, for example have documented the deregulation of the IL-23/IL-17 pathway in a wide variety of chronic inflammatory and auto-immune diseases, including rheumatoid arthritis, psoriasis, inflammatory bowel disease (IBD), multiple sclerosis, and ankylosing spondylitis. As a non-limiting example, the frequency of Th17 cells and the levels of Th17-related cytokines have been found to be elevated in subjects with atherosclerosis, with Th17-related cytokines correlated with the severity of carotid artery plaques (Zhang et al., 2013, Liu et al., 2012), multiple sclerosis (Li et al., 2011), type 1 diabetes (Ryba-Stanislawowska et al., 2013), rheumatoid arthritis (Zizzo et al., 2011), ankylosing spondylitis (Xueyi et al., 2013), systemic lupus erythematosus (Wong et al., 2008), etc.

Thus, the modulation of the IL-23 and/or downstream IL-17 cytokines is of major interest in improving treatment of auto-immune diseases and/or inflammatory conditions.

Recently, various monoclonal antibodies neutralizing the IL-23 or IL-17A have been shown to be effective in treating human auto-immune and inflammatory diseases such as psoriasis, rheumatoid arthritis, and Crohn's disease, indicating that the IL-23/IL-17-mediated inflammatory cascade is indeed associated with the pathogeny of such diseases and that therapies specifically targeting these cytokines may be envisaged. As an example, several monoclonal antibodies targeting the p19 subunit of IL-23, including guselkumab, risankizumab, and tildrakizumab, have completed phase III clinical trials for the treatment of moderate-to-severe psoriasis (Puig, 2019, Haugh et al., 2018, Kolli et al., 2019).

However, while their efficacy has been proven, cost remains a major problem associated with the use of monoclonal antibodies, in particular for diseases which require long-term treatment. In addition, depending on the medical history of a given individual, administration of monoclonal antibodies may not be recommended. For example, subjects suffering from depression or having suicidal ideation are advised to avoid taking brodalumab, an agonist of the IL-17R (Foulkes et al., 2019). IL-17 agonists are also associated with exacerbation or declaration of de novo cases of IBD (Wang et al., 2018). Various adverse events have also been associated with administration of such antibodies including nasopharyngitis, headache, upper respiratory tract infection, which may not make them suitable for all subjects. An increased risk of activation of latent tuberculosis has also been described. Furthermore, treatment with monoclonal antibodies such as guselkumab should be interrupted for 12 weeks prior to the reception of any live viral or bacterial vaccine, as detailed in the product leaflet issued by the British Association of Dermatologists; thus, in these cases, continuous treatment is not possible. There also remains a risk of neutralizing antibodies being generated against these monoclonal antibodies, which may reduce their efficacy or result in adverse events such as infusion reactions and/or immune complex formation. The long-term safety of these agents remains to be determined. Finally, it should be noted that these agents are typically administered via subcutaneous injection, which is not adapted to all subjects, and which may furthermore need to be performed in a hospital setting (e.g., in cases of large injection volumes, when home-administration is associated with a risk of under- or over-dosing, or if self-administration is not approved by regulatory health administrations).

Thus, there exists a need for new products and methods targeting IL-23 and, consequently, downstream IL-23-controlled pathways, which may be used in the prevention and/or treatment of diseases, in particular, the prevention and/or treatment of autoimmune and/or inflammatory diseases in humans. There further remains a need for new products that are available at low cost. Preferably, said products and methods may be administered over a long period of time (e.g., weeks or months) or at frequent intervals (e.g., daily), have few side effects, and/or need not necessarily be administered by injection.

### SUMMARY OF THE INVENTION

The present invention is directed to a composition comprising 2′,4′-dihydroxy-3′,6′-dimethoxychalcone (“2,4-D”) or a pharmaceutically acceptable salt thereof for use as a medicament. The present invention is more particularly directed to a composition comprising 2,4-D or a pharmaceutically acceptable salt thereof for use in the prevention and/or treatment of a disease associated with IL-23 expression, except cancers.

Indeed, the inventors have surprisingly found that 2,4-D specifically inhibits IL-23 production/release and the activation of downstream IL-23/IL-17 pathways. As is notably illustrated in the examples below, 2,4-D surprisingly strongly inhibits IL-23 gene expression, as shown in a cell line expressing the IL-23 gene, thereby inhibiting IL-23 cytokine production. Furthermore, the inventors have surprisingly shown here that the administration of 2,4-D in accepted animal models for auto-immune and/or inflammatory diseases (e.g., psoriasis, rheumatoid arthritis, acute gout arthritis, graft rejection, endometriosis) as well as degenerative diseases (e.g., amyotrophic lateral sclerosis, macular degeneration), inhibited IL-23 expression and resulted in the successful treatment of these pathologies.

2,4-D preferably comprises at least 90% of the (E) isomer or a pharmaceutically acceptable salt thereof. More preferably, 2,4-D consists essentially of the (E) isomer or a pharmaceutically acceptable salt thereof. Indeed, the inventors have surprisingly found that the (E) isomer of 2,4-D more strongly inhibits IL-23 expression than the (Z) isomer.

Preferably, said composition further comprises a pharmaceutically acceptable carrier.

Preferably, said composition further comprises at least one additional therapeutic agent. Alternatively, 2,4-D or said pharmaceutically acceptable salt thereof is advantageously the sole therapeutic agent in said composition.

Preferably, said composition is administered via a local, enteral, or parenteral route, more preferably via a topical, oral, intraperitoneal, intravenous, intradermal, subcutaneous, intra-articular, or mucosal route, more preferably via the oral or intraperitoneal route.

Preferably, said composition is administered at least once per day.

Preferably, 2,4-D or said pharmaceutically acceptable salt thereof is administered at a dose comprised between 1 mg/kg and 10 mg/kg per day.

Preferably, the invention is directed to a composition comprising 2,4-D or a pharmaceutically acceptable salt thereof for use in the treatment of a disease associated with IL-23 expression, wherein said disease is selected from an autoimmune disease and/or an inflammatory disease, a neurodegenerative disease other than Alzheimer's. Said disease associated with IL-23 expression may be a systemic or localized disease.

Preferably, said autoimmune disease is selected from rheumatologic autoimmune diseases, gastrointestinal and liver autoimmune/inflammatory diseases, vasculitis, renal autoimmune diseases, dermatological autoimmune diseases, hematologic autoimmune diseases, atherosclerosis, uveitis, autoimmune ear diseases, Raynaud's syndrome, diseases associated with organ transplantation and autoimmune endocrine diseases such as type-1 diabetes.

Preferably, said disease associated with IL-23 expression is:
an inflammatory disease, wherein said inflammatory disease is selected from arthritis, rheumatoid arthritis, acute gout arthritis (also herein referred to as “gout” or “gout crisis”, i.e., not the persistently elevated levels of uric acid in the blood), osteoarthritis, an inflammatory bone disease; an inflammatory lung diseases, preferably asthma; Behcet's disease; an inflammatory disease of the eye preferably retinal diseases comprising inflammatory processes, uveitis; a chronic inflammatory disease of the gums preferably gingivitis or periodontitis; an inflammatory disease of the kidney preferably a uremic complication, glomerulonephritis or nephrosis; an inflammatory disorder of the skin; a chronic demyelinating or degenerative diseases of the nervous system; Parkinson's disease; Huntington's disease; amyotrophic lateral sclerosis; an immune-complex vasculitis; systemic lupus erythematosus (SLE); an inflammatory disease of the heart, preferably cardiomyopathy, coronary thrombosis, ischemic heart disease hypercholesterolemia, or atherosclerosis; preeclampsia; schizophrenia; chronic liver failure, or brain or spinal cord trauma; endometriosis; and/or a neurodegenerative disease wherein said neurodegenerative disease is selected from among migraine headache, trigeminal neuralgia, myasthenia gravis, a demyelinating disease, preferably acute transverse myelitis; an extrapyramidal and cerebellar disorder; a hyperkinetic movement disorder, preferably Huntington's Chorea or senile chorea; a drug-induced movement disorder, preferably a disorder induced by drugs which block CNS dopamine receptors; a hypokinetic movement disorder, preferably Parkinson's disease; progressive supranuclear Palsy; a structural lesion of the cerebellum; a spinocerebellar degenerations, preferably spinal ataxia, Friedreich's ataxia, a cerebellar cortical degeneration, or a multiple systems degeneration; a systemic disorder, preferably Refsum's disease, abetalipoproteinemia, ataxia, telangiectasia, or mitochondrial multi-system disorder; a disorder of the motor unit, preferably a neurogenic muscular atrophy; Down's Syndrome in middle age; Diffuse Lewy body disease; Senile Dementia of Lewy body type; Wernicke-Korsakoff syndrome; chronic alcoholism; Creutzfeldt-Jakob disease; Subacute sclerosing panencephalitis, Rasmussen's encephalitis, Huntington's disease, Hallerrorden-Spatz disease; Dementia pugilistica; neurotraumatic injury; inflammatory pain; autism; depression; head trauma, stroke; cognitive disorders; epilepsy; and macular degeneration; and/or
a non-infectious inflammatory respiratory disease wherein said non-infectious respiratory disease is selected from the group consisting of, interstitial lung disease, cystic fibrosis, pulmonary oedema, chronic obstructive pulmonary disease, asthma, allergic alveolitis, restrictive lung disease, fibrosis, lupus erythematosus, systemic scleroderma or sarcoidosis, and rhinitis.

Preferably, said disease associated with IL-23 expression is selected from graft-versus-host disease (GvHD), psoriasis, rheumatoid arthritis, gout crisis, amyotrophic lateral sclerosis, macular degeneration and endometriosis.

Another aspect of the present invention is to provide a method for preventing and/or treating a disease associated with IL-23 inappropriate expression, except cancers, in a subject in need thereof, comprising administering an effective amount of the composition as provided herein to said subject.

### FIGURES

The present invention is illustrated by the following figures.

FIG. 1: Suppression of IL-23 gene expression by 2,4-D in LPS-stimulated THP-1 cell culture.

FIG. 2: Suppression of IL-23 production by 2,4-D in LPS-stimulated THP-1 cell culture.

FIG. 3: Suppression of IL-23 production by the (E) and (Z) isomers of 2,4-D in LPS-stimulated THP-1 cell culture.

FIG. 4: Suppression of IL-23 production in LPS-stimulated THP-1 cell culture by natural or synthetic 2,4-D.

FIG. 5: Suppression of IL-23 production in LPS-stimulated THP-1 cell culture by 2,4-D as compared to other compounds. IBC: Isobavachalcone, HMC: 4′-hydroxy-2′6′-dimethoxychalcone, 2′-HC: 2′-hydroxychalcone, 4-HC: 4-hydroxychalcone, 2′,2-DHC: 2′,2-dihydroxychalcone, 2′,3-DHC: 2′,3-dihydroxychalcone, 2′,4-DHC: 2′,4-dihydroxychalcone, 2′,4′-DHC: 2′,4′-dihydroxychalcone 2′,5′-DHC: 2′,5′-dihydroxychalcone, and 2′,4′4-THC: 2′,4′,4-trihydroxychalcone, TFM: 2-trifluoromethyl-2′-methoxychalcone, HYA: Hydroxysafflor yellow A.

FIG. 6: Use of 2,4-D in the treatment of rheumatoid arthritis in the mouse model. (A) Joint inflammation in forelimbs with increasing concentrations of 2,4-D. (B) Joint inflammation in hindlimbs with increasing concentrations of 2,4-D. (C) Mean clinical score over time with 2,4-D treatment.

FIG. 7: IL-17 and IL-23 gene expression in human synovial cells collected from human subjects suffering from rheumatoid arthritis in cultures comprising increasing concentrations of 2,4-D.

FIG. 8: Levels of (A) IL-23 and (B) IL-17A cytokines present in the supernatant of human synovial cells collected from human subjects suffering from rheumatoid arthritis in cultures comprising increasing concentrations of 2,4-D.

FIG. 9: Representative illustration of the inflammation observed in the 12-O-Tetradecanoyl Phorbol-13-Acetate (TPA)-induced psoriasis mouse model (right panel) as compared to a control mouse (left panel).

FIG. 10: Effect of increasing concentrations of 2,4-D on hyperplasia in the TPA-induced psoriasis mouse model.

FIG. 11: Effect of increasing concentrations of 2,4-D on epidermal thickness in the TPA-induced psoriasis mouse model.

FIG. 12: Balb/c to C57BL/6 skin graft survival. Treatment with oral 2,4-D (2, 10 or 50 mg/kg of daily 2,4-D, bid).

FIG. 13: IL-23-mediated inhibition of IL-17 production in anti-CD3/CD28-stimulated mouse spleen cells.

FIG. 14: IL-23 production in LPS-stimulated THP-1 cells in the presence of 2,4-D as compared to nonsteroidal anti-inflammatory drugs (NSAIDs).

FIG. 15: Fold change of TNF/IL-17-signaling gene expression in LPS-stimulated TPH-1 cells treated with 2,4-D, RPM, or dexamethasone (DEX).

FIG. 16: Production of TNF/IL-17-signaling cytokines and chemokines in culture supernatants of LPS-stimulated TPH-1 cells treated with 2,4-D, RPM, or DEX.

FIG. 17: Change in MSU-induced knee joint edema in mice treated with high and low doses of anti-inflammatory drugs. CLC: colchicine.

FIG. 18: Measurement of MSU-induced leukocyte recruitment to the synovial cavity of knee joint in mice treated with different anti-inflammatory drugs (100 mg/kg 2,4-D, 1 mg/kg CLC, or 0.5 mg/kg DEX).

FIG. 19: MSU-induced pro-inflammatory cytokines in the knee joint tissue of mice pretreated with different anti-inflammatory drugs (100 mg/kg 2,4-D, 1 mg/kg CLC, or 0.5 mg/kg DEX).

FIG. 20: Kaplan-Meier analysis estimating the age at which a mouse cannot perform a hip extension during the tail suspension test (Male SOD1 G93A Vehicle, n=8; vs. Male SOD1 G93A 2,4-D, n=9; Mantel-Cox test, P<0.05).

FIG. 21: Kaplan-Meier analysis estimating the age at which a mouse cannot perform a back extension during the tail suspension test (Male SOD1 G93A Vehicle, n=8; vs. Male SOD1 G93A 2,4-D, n=9; Mantel-Cox test, P<0.05).

FIG. 22: Evolution of the Neuroscore (NS) in function of the age of mice. NS1 is reached when a mouse cannot keep hindlimbs extended away from lateral midline for more than 2 seconds. NS2 is reached when the toes curl downwards at least twice during a 75 cm. NS3 is reached when at least one hindlimb shows rigid paralysis or minimal joint movement during the suspension test, or the walking test. Mice are euthanized when they reach NS3. (Male SOD1 G93A Vehicle, n=8; vs. Male SOD1 G93A 2,4-D, n=9; Mixed-effects model, P=0.06).

FIG. 23: Kaplan-Meier analysis estimating the age at which a mouse reaches the NS3 (Male SOD1 G93A Vehicle, n=8; vs. Male SOD1 G93A 2,4-D, n=9; Mantel-Cox test, P<0.05).

FIG. 24: Repartition of impacts by leakage intensity grading in the different groups of treatment (expressed in %, n=43 in vehicle, 53 in TA and 38 in 2,4-D (New treatment, NT) groups); fused impacts were excluded from grading.

FIG. 25: Statistical analysis showed that 2,4-D (New T) significantly reduced the grading score of impacts as compared to both vehicle and TA.

FIG. 26: FA images and infrared images of CNV in vehicle, TA and 2,4-D (New T) groups.

FIG. 27A: Surface (μm2) (mean±SD) of CNV in 2,4-D (New T) and vehicle groups.

FIG. 27B: Representative image of lectin-FITC labelled CNV (upper panel) and IBA-1 stained monocytes (lower panel).

FIG. 28: experimental protocol for endometriosis model.

FIG. 29: cytokine concentration measurement in cell-free peritoneal lavage collected from endometriosis model, with no EDT (white), EDT+vehicle (light grey) and EDT+2,4-D (dark grey).

FIG. 30: phenotypic study of immune cells from the peritoneal cavity collected from endometriosis model, with no EDT (white), EDT+vehicle (light grey) and EDT+2,4-D (dark grey).

FIG. 31: cell surface marker expression of immune cells from the peritoneal cavity collected from endometriosis model, with no EDT (white), EDT+vehicle (light grey) and EDT+2,4-D (dark grey).

FIG. 32: Schema illustrating a mechanism of action of 2,4-D.

### DETAILED DESCRIPTION OF THE INVENTION

Before describing the present invention in detail, it is to be understood that the invention is not limited to particularly exemplified aspects and may, of course, vary. It is also to be understood that the terminology used herein is for the purpose of describing particular embodiments of the invention only, and is not intended to be limiting.

All publications, patents and patent applications cited herein, whether supra or infra, are hereby incorporated by reference in their entirety.

### I. Composition

A pharmaceutical composition comprising 2′,4′-dihydroxy-3′,6′-dimethoxychalcone or a pharmaceutically acceptable salt thereof is provided herein. A composition comprising 2′,4′-dihydroxy-3′,6′-dimethoxychalcone or a pharmaceutically acceptable salt thereof for use as a medicament is further provided herein.

The present invention is further directed to a composition comprising 2′,4′-dihydroxy-3′,6′-dimethoxychalcone or a pharmaceutically acceptable salt thereof for use in the prevention and/or treatment of a disease associated with IL-23 expression, wherein said composition comprises 2′,4′-dihydroxy-3′,6′-dimethoxychalcone or a pharmaceutically acceptable salt thereof. Advantageously, said composition down-regulates expression of the IL-23 gene. This down-regulation is highly advantageous as it specifically reduces or even completely prevents IL-23 mRNA transcription and downstream protein translation, thereby minimizing and even abolishing the effects associated with the presence of the IL-23 protein, such as the activation of the IL-23/IL-17 pathway.

The molecule “2′,4′-dihydroxy-3′,6′-dimethoxychalcone,” also referred to interchangeably herein as “2,4-D,” has the CAS number 54299-50-2 and the chemical formula C₁₇H₁₆O₅. Unless explicitly indicated, reference to “2′,4′-dihydroxy-3′,6′-dimethoxychalcone” or “2,4-D” herein also refers to any pharmaceutically acceptable salt thereof. While 2,4-D may be comprised of a mixture of (E) and (Z) isomers, 2,4-D preferably comprises a majority of the (E) isomer, preferably at least 60%, 70%, 80%, or 90%, more preferably at least 95%, 96%, 97%, 98%, even more preferably at least 99% of the (E) isomer or a pharmaceutically acceptable salt thereof. According to a particularly preferred embodiment, 2,4-D consists essentially of the (E) isomer or a pharmaceutically acceptable salt thereof. The (E) isomer more particularly has the CAS number 129724-43-2. Indeed, the inventors have surprisingly found that the (E) isomer of 2,4-D inhibits IL-23 production more strongly than the (Z) isomer in an LPS stimulated THP-1 cell line.

Any disease or condition that may benefit from a treatment with the composition comprising 2,4-D or a pharmaceutically acceptable salt thereof as provided herein is comprised within the meaning of the term “disease.” The terms “disease associated with IL-23 expression” and “IL-23 associated disease” are used herein interchangeably and refer to any disease or condition, except cancers, in which IL-23 activity contributes directly or indirectly to the disease or condition, in particular a disease or condition except cancers in which IL-23 is abnormally expressed.

The term “IL-23 gene” as used herein refers to the gene coding for the p19 subunit of the IL-23 heterodimer. Similarly, the term “IL-23 protein” as used here refers to the p19 subunit of the IL-23 heterodimer. Indeed, only this subunit is specific for IL-23, as the p40 subunit is comprised in both the heterodimer forming IL-12 as well as the heterodimer forming IL-23. Preferably, the IL-23 protein has the sequence of SEQ ID NO: 1. Preferably, the IL-23 mRNA has the sequence of SEQ ID NO: 2. Preferably, the IL-23 gene has the nucleotide sequence of SEQ ID NO: 3. In some cases, the IL-23 gene or protein may alternatively be referred to as “IL-23A” or “IL23p19”.

The expressions “down-regulating gene expression” or “inhibiting gene expression” as used herein are used interchangeably and refer to a measurable or observable reduction in gene expression or a complete abolition of detectable gene expression at the level of the mRNA product transcribed from the IL-23 target gene and/or the protein product resulting from translation of the mRNA product, or at the phenotypic level. Down-regulation of gene expression can be confirmed by measurement of mRNA or protein expression using molecular techniques such as RNA solution hybridization, PCR, nuclease protection, Northern hybridization, reverse transcription, gene expression monitoring with a microarray, antibody binding, enzyme linked immunosorbent assay (ELISA), Western blotting, radioimmunoassay (RIA), other immunoassays, or fluorescence activated cell analysis (FACS), or by phenotypic analysis of a subject (e.g. a measurable or observable reduction of an IL-23-associated disease state, such as a reduction in symptom severity or the elimination of a symptom). According to a preferred method, IL-23 down-regulation is observed by measuring IL-23 protein levels in serum, more preferably via ELISA. Advantageously, the down-regulation of IL-23 gene expression inhibits or in some cases even shuts down the IL-23/IL-17 biological pathway.

According to a further aspect of the present invention, IL-23 gene expression is down-regulated in at least one cell selected from antigen presenting cells (APCs), epithelial cells, keratinocytes, synoviocytes, and podocytes.

The down-regulatory effect of 2,4-D on IL-23 gene expression may be calculated as being a reduction of at least 30%, 40%, 50%, 60%, preferably at least 70%, at least 80% or even more preferably at least 90% or at least 95% when compared with IL-23 gene expression in an untreated diseased subject. In the present context, IL-23 gene expression is preferably undetectable or at low levels in healthy control subjects. Preferably, the down-regulatory effect of 2,4-D on IL-23 gene expression reduces IL-23 such that it is at least equivalent to, or lower than, the level of IL-23 detected in a healthy subject. The level of IL-23 gene expression observed in healthy subjects is lower than the expression level that is observed in diseased subjects. As a non-limiting example, serum IL-23 levels in healthy children of less than 5 years of age have been shown to average approximately 14 pg/mL (Alyasin et al., 2017). Serum IL-23 levels in healthy adults between ages 25 and 47 have been reported to average approximately 15.5 pg/mL according to Zihni Bilik et al., 2016, while serum IL-23 levels in a different sampling of healthy adults of age 39.5±12.7 years (range: 18-65 years) have been reported to average 37.7±15.6 pg/ml (Alsheikh et al., 2019). Thus, according to a preferred embodiment, administration of 2,4-D reduces serum IL-23 level to an average of 14 pg/mL or lower in children of less than 5 years of age, or to an average of 38 pg/mL or lower in adults. Of course, the skilled person is able to determine IL-23 levels in healthy versus diseased patients for any particular cohort, and evaluate the effect of 2,4-D on reducing elevated IL-23 levels in diseased subjected such that the disease state is improved.

An improvement in disease state may furthermore be observed in diseased patients even when IL-23 levels remain above those observed in healthy subjects if this level is decreased as compared to the level of IL-23 present before treatment with 2,4-D. Thus, according to a preferred embodiment, the down-regulatory effect of 2,4-D on IL-23 gene expression reduces IL-23 levels in a subject such that it is lower than the level of IL-23 detected in said subject prior to treatment with 2,4-D. Down-regulation or inhibition of IL-23 gene expression is “specific” when down-regulation or inhibition of said gene occurs without manifest effects on other genes of a cell.

The term “treating” or “treatment” as used herein refers to an alleviation of symptoms associated with a disorder or disease associated with IL-23 expression, or inhibition of further progression or worsening of said symptoms of said disease or disorder. The term “prevention” as used herein refers to the prevention or prophylaxis of the disease or disorder associated with IL-23 expression. The terms “treatment” and “prevention” as used herein are not meant to be exclusively absolute terms. Indeed, the term “prevention” as used herein may refer to a delay in the appearance of symptoms, a reduction in symptom severity upon appearance, or a reduction in the frequency of disease episodes.

Similarly, the term an “effective amount” as used herein in the context of a therapeutic agent such as 2,4-D, or a “therapeutically effective amount” of a therapeutic agent such as 2,4-D refers to an amount of the agent that alleviates, in whole or in part, symptoms associated with the disorder or condition, or halts or slows further progression or worsening of those symptoms, or prevents or provides prophylaxis for the disorder or condition. In particular, an “effective amount” refers to an amount effective, at dosages and for periods of time necessary, to achieve the desired therapeutic result. A therapeutically effective amount may notably be administered in one or more administrations. A therapeutically effective amount is also one in which any toxic or detrimental effects of compounds of the invention are outweighed by the therapeutically beneficial effects.

The term “pharmaceutically acceptable salt” as used herein includes salts of the compounds of the present invention derived from the combination of such compounds and an organic or inorganic acid. In practice the use of the salt form is substantially equivalent to use of the base form. The compounds of the present invention are useful in both free base and salt form, with both forms being considered within the scope of the present invention.

Pharmaceutically acceptable salts are non-toxic salts at the concentration at which they are administered. The preparation of such salts can facilitate the pharmacological use by altering the physical-chemical characteristics of the composition without preventing the composition from exerting its physiological effect. Examples of useful alterations in physical properties include lowering the melting point to facilitate transmucosal administration and increasing the solubility to facilitate the administration of higher concentrations of the drug.

Pharmaceutically acceptable salts include acid addition salts such as those containing sulfate, hydrochloride, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethane sulfonic acid, benzene sulfonic acid, p-toluenesulfonic acid, cyclohexyl sulfamic acid, and quinic acid.

Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

### II. Formulation

The composition or composition for use according to the invention may further comprise at least at least one pharmaceutically acceptable carrier. The term “pharmaceutically acceptable carrier” or “pharmaceutically acceptable carrier” are used interchangeably herein and refer to a component, or combination of components, that is compatible with the composition, does not generate unwanted side-effects in the subject, and that is generally considered to be non-toxic. A pharmaceutically acceptable carrier is most commonly implicated in facilitating administration of the composition, increasing product shelf-life or efficacy, or improving the solubility or stability of the composition. The pharmaceutically acceptable carrier is generally considered to be pharmacologically inactive. However, in some cases, the excipient itself may also have a therapeutic effect, for example an adjuvant effect.

Pharmaceutically acceptable carriers or excipients are well-known in the prior art and can easily be adapted by the skilled person based on the desired galenic formulation of the composition. As a non-limiting example, said pharmaceutically acceptable carrier may comprise one or more antioxidants, buffers, bulking substances, suspending agents, solubilizing agents, matrix forming additives, humectants, diluents, solvents, plasticizers, oily/emulsifying/aqueous bases, gelling agents, preservatives, tonicity adjusting agents, vehicles, and stabilizers. As a non-limiting example, the composition may comprise at least one pharmaceutically acceptable carrier to approximate physiological conditions, such as a pH buffering agent. Useful buffers include for example, sodium acetate/acetic acid buffers. The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions. Certain additional pharmaceutically acceptable carriers are provided below (see section III).

The composition for use may be provided in the form of a liquid, solid, semi-solid, gel, or in any suitable matrix. In a preferred embodiment, said composition is suspended or dissolved in an aqueous carrier, such as an isotonic buffer solution.

Preferably, the composition is sterilized by conventional sterilization techniques, or sterile filtered.

The galenic formulation, method of administration, and dosage can further be determined based on widely-accepted criteria for adapting patient treatments, including their general health, age, weight, tolerance to treatment, etc., as necessary. It may also depend on the degree, severity and type of disease.

According to one aspect of the invention, the composition for use provided herein comprises 2,4-D or said pharmaceutically acceptable salt thereof as the sole active agent in said composition.

According to an alternative aspect, the composition for use provided herein may further comprise at least one additional therapeutic agent. A “therapeutic agent,” as provided herein, is a medicinal compound or mixture thereof that is effective and medically indicated for the prevention and/or treatment of a disease in a subject. As a non-limiting example, said at least one therapeutic agent may be selected from the group consisting of a TNF antagonist (e.g., a TNF chemical or protein antagonist, a TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-I or TBP-II), nerelimonmab, infliximab, eternacept (Enbrel™), adalimulab (Humira™), CDP-571, CDP-870, afelimomab, lenercept, and the like), an anti-rheumatic (e.g., methotrexate, auranofin, aurothioglucose, azathioprine, gold sodium thiomalate, hydroxychloroquine sulfate, leflunomide, sulfasalzine), a muscle relaxant, a narcotic, a non-steroid anti-inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, such as antiviral interferon, chemokine receptor antagonist, integrase strand transfer inhibitor, NNRTI, NS5A inhibitor, nucleoside reverse transcriptase inhibitor (NRTI), protease inhibitor, or purine nucleoside, a carbapenem, cephalosporin, a fluororquinolone, a macrolide, a penicillin, a sulfonamide, a tetracycline, another antimicrobial), an antipsoriatic, a corticosteriod, an anabolic steroid, a diabetes-related agent, a mineral, a nutritional, a flavonoid, a thyroid agent, a vitamin, a calcium related hormone, an antidiarrheal, an antitussive, an antiemetic, an antiulcer, an immunotherapeutic agent, a laxative, an anticoagulant, an erythropoietin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist such as an IL-17 antagonist or a second IL-23 antagonist, and/or and any combination of two or more thereof. Preferably when the composition comprises 2,4-D and a second IL-23 antagonist, said antagonist has a different mechanism of action (e.g., blocking IL-23 protein binding to the IL-23R receptor). When the composition of the present invention is used in the treatment of a specific disease, preferably it is administered in combination with one or more molecules as provided above according to an established first-line or second-line therapy for treating said disease.

Said therapeutic agent may be administered either simultaneously, or separately, or sequentially to the composition comprising 2,4-D. Thus, a further object of the present invention is a composition for use in the treatment of IL-23 associated diseases comprising 2,4-D as provided herein, which is used in combination with at least one therapeutic agent. Preferably, 2,4-D is simultaneously, sequentially, or separately administered with the therapeutic agent(s). The composition for use as a medicament, preferably for use in the treatment of IL-23 associated diseases comprising 2,4-D, which is used in combination with at least one therapeutic agent, encompasses all of the modalities as described and/or defined herein.

The combined effect of 2,4-D and the at least one therapeutic agent may advantageously improve treatment efficacy (i.e., when the dose of therapeutic agent is maintained, an improved effect, such as a reduction in the severity of a symptom, the number of symptoms, or frequency of relapse, can be observed) in an additive or synergistic manner. Advantageously, dosage of the therapeutic agent may be reduced without negatively influencing therapeutic effects. Accordingly, side effects associated with therapeutic agent administration may also be reduced. Advantageously, new population groups for which therapeutic agents are not effective, or who suffer from side effects at the administered dose of therapeutic agent, can be treated, as lower doses of therapeutic agents can be administered when in combination with 2,4-D. In some cases, the frequency of administration of therapeutic agent treatment may also advantageously be reduced or even stopped. As a particular example, when the therapeutic agent is administered daily, the frequency of use may advantageously be reduced e.g., every other day, when the composition comprising 2,4-D is also administered according to any of the modalities provided herein.

### III. Administration of the Composition

According to a further aspect of the invention, the composition or composition for use provided herein is administered to a subject via a local, enteral, or parenteral route. In particular, the composition may be administered locally via topical application, for example to the skin and/or mucosal surfaces, more particularly to a lesion, via local infusion during surgery (for example perfusing a tissue or organ prior to grafting), or via transdermal administration.

Administration via the topical route to the skin and/or mucosal surfaces is particularly preferred when treating autoimmune dermatological disorders such as psoriasis, dermatitis, or other localized inflammatory reactions of the skin and/or mucosal surfaces.

Mucosal surfaces are preferably selected from nasal, tracheal, gingival, pharyngeal, bronchial, rectal, preputial and vaginal membranes. In the present context, the mucosal surface also includes the external surface of the eye, i.e., the mucosa of and surrounding the eye. Administration to the bronchial, bronchiolar, tracheal, pharyngeal, nasal, oral, gingival, preputial or pharyngeal mucosa can be obtained by formulating the pharmaceutical composition as an inhalable, as a spray, and the like (e.g., nasal spray, aerosol spray or pump spray and the like), as a solution, as a gel, etc. Nebulizer devices suitable for delivery of pharmaceutical compositions to the nasal mucosa, trachea and bronchioli are well-known in the art. The composition of the present invention may be administered to the skin and/or mucosal surfaces in the form of a patch, gel, cream, lotion, ointment, film or salve. Advantageously, the viscosity or texture of the composition is modulated to improve application or efficacy (e.g., contact time). As a particular example, the pharmaceutical composition may comprise a vehicle selected in the group comprising solutions, emulsions, microemulsions, oil-in-water emulsions, anhydrous lipids and oil-in-water emulsions, other types of emulsions in view of administration to one or more of the mucosal surfaces described above.

The composition of the present invention may be administered more specifically to the rectal or vaginal mucosa as a solution, enema, foam, or suppository. Preferred vehicles for rectal or vaginal delivery include hydrophilic and hydrophobic vehicles such as those commonly used in formulating emulsion or gel preparations (e.g., oil/water emulsion gel).

According to another aspect, the composition may be administered enterally via the oral route. The term “oral route” as used herein comprises orogastric administration, in which administration is tracheal, pharyngeal, esophageal, gastric, duodenal, or intestinal as well as oromucosal administration, including buccal, sublingual, or sublabial administration.

Administration to the digestive tract can be obtained by formulating the composition as an aqueous suspension, elixir, lozenge, tablet, syrup, capsule or microcapsule, or any other formulation appropriate for digestive delivery. As an example, microcapsules for oral administration are disclosed in the PCT application WO 2007/140613. Alternatively, digestive delivery may be performed by incorporating the composition into an appropriate liquid and/or foodstuff, such as beverages, yogurts, etc. for consumption. For solid compositions (e.g., tablet compositions), pharmaceutically acceptable carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc., 2,4-D or a pharmaceutically acceptable salt thereof as described herein in a pharmaceutically acceptable carrier, such as water, saline, aqueous dextrose, glycerol, ethanol, and the like, thereby forming a solution or suspension. If desired, the pharmaceutical composition to be administered may also contain minor amounts of pharmaceutically acceptable carriers such as wetting or emulsifying agents, pH buffering agents and the like, for example, sodium acetate, sorbitan mono-laurate, triethanolamine acetate, triethanolamine oleate, etc. Methods of preparing such dosage forms are known, or will be apparent, to those skilled in the art. See, e.g., Remington: The Science and Practice of Pharmacy, 2012, 22^{nd} edition.

For oral administration, the composition will generally take the form of a tablet or capsule, or may be an aqueous or nonaqueous solution, suspension or syrup. Tablets and capsules are preferred oral administration forms. Tablets and capsules for oral use will generally include one or more commonly used carriers such as lactose and corn starch. Lubricating agents, such as magnesium stearate, are also typically added. When liquid suspensions are used, the active agent may be combined with emulsifying and suspending agents. If desired, flavoring, coloring and/or sweetening agents may be added. Other pharmaceutically acceptable carriers for incorporation into an oral formulation include preservatives, suspending agents, thickening agents, and the like.

The enteral route is particularly advantageous as delivery is easy and convenient for subjects and induces a systemic effect.

According to another aspect, the composition provided herein is administered via the parenteral route, such as via intramuscular (IM), intravenous (IV), intraarterial, intra-articular, intracardiac, intraocular, intraosseous, intraperitoneal (IP), subcutaneous (SC), intracerebral, intraventricular, intracerebroventricular, intradermal (ID), intraepithelial or intrathecal administration. More preferably, the composition is administered via the IM, IV, ID, SC, or intra-articular route. Such routes of administration are well-known in the art. As an example, intradermal administration may be performed with needle-devices such as those disclosed in U.S. Pat. No. 6,933,319 and in PCT application WO 2004/101025, or with appropriate needle-free devices. Compounds useful in the invention can notably be provided for parenteral administration via injection, infusion, or implant. Typically, when administering the composition, materials are selected which do not interact with 2,4-D, or a therapeutic agent when present in combination with 2,4-D. Preferably, 2,4-D does not absorb to the selected material.

In one aspect, the composition provided herein may be administered directly to a site (or former site) of a manifestation of an autoimmune disease (e.g., a lesion or plaque), preferably via a method of direct injection (e.g., intra-articular administration in the case of treating a disease having localized articular inflammation) or direct application (e.g., topical administration). In another aspect, the composition provided herein may be administered directly to the central nervous system, preferably via intraventricular or intrathecal injection. This is particularly advantageous when treating neurodegenerative diseases associated with IL-23 expression. Intraventricular injection may notably be facilitated by an intraventricular catheter, for example, attached to a reservoir.

Preferably the composition provided herein is administered via a topical, oral, intraperitoneal, intravenous, intradermal, subcutaneous, or intraarticular route. More preferably, the composition provided herein is administered via an oral or intraperitoneal route, even more preferably via the intraperitoneal route. In some cases, it is advantageous to employ a multi-route regimen, for example when treating a subject having two or more different diseases associated with IL-23 or when said composition is administered in combination with a therapeutic agent as provided herein (e.g., the composition provided herein may be administered orally in combination with another agent administered intravenously).

The composition containing 2,4-D is preferably in a unit dosage form suitable for single administration of a precise dosage or in multi-dose containers with unit metering capability for the chosen route of administration. The composition provided herein may furthermore be administered as an infusion (i.e., over time, for example for a duration equal or superior to 15 minutes, 30 minutes, or even 60 minutes) or as a bolus (i.e., as a single administration having a duration inferior to 15 minutes, 5 minutes or even 1 minute). Alternatively, the composition may be administered in a controlled-release formulation. As a non-limiting example, said controlled-release formulation may comprise matrices of solid hydrophobic polymers containing 2,4-D or the pharmaceutically acceptable salt thereof, which matrices are in the form of shaped articles, e.g., films, or microcapsules. Examples of controlled-release matrices include polyesters, hydrogels (for example, poly(2-hydroxyethyl-methacrylate), or poly(vinylalcohol), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the injectable microspheres composed of lactic acid-glycolic acid copolymer and leuprolide acetate), and poly-D-(−)-3-hydroxybutyric acid. In some cases, the composition provided herein may be administered by pulse infusion. In one aspect, the dosing is given by injections, most preferably intravenous or subcutaneous injections, depending in part on whether the administration is to be brief or chronic.

For the prevention and/or treatment of IL-23 associated disease, the appropriate dosage of the composition provided herein will depend on a variety of factors such as the specific disease to be treated, for example selected among those defined herein, the severity and course of the disease, whether the composition is administered for preventive or therapeutic purposes, previous therapies that have been administered, the site of delivery of the agent, the method of administration, the subject's clinical history, the subject's response to the composition, which mammal is being treated in cases where the subject is not human, etc. The ranges of effective doses provided below are not intended to limit the invention and represent preferred dose ranges.

Depending on the type and severity of the disease, about 1 mg/kg to 10 mg/kg (e.g., 1-10 mg/kg) of 2,4-D is an initial candidate dosage for administration to a subject, whether, for example, by one or more separate administrations. A typical daily dosage might range from about 1 mg/kg to 5 mg/kg or more, depending on the factors mentioned above. For repeated administrations over several days or longer, depending on the condition, the treatment is sustained until a desired lessening of one or more disease symptoms occurs. However, other dosage regimens may be useful. The progress of this therapy is easily monitored by conventional techniques and assays. Thus, according to a preferred embodiment, a composition comprising 1-10 mg/kg of 2,4-D, more preferably 1-5 mg/kg, of 2,4-D or a pharmaceutically acceptable salt thereof is administered to a subject per day, in one or more administrations.

In some cases, the composition provided herein may be administered to a subject one or more times daily (e.g. one, two, or three times, for example prior to, during, or after meals) and can be administered for several consecutive days (e.g. for one, two, three, four, five, six, seven, eight, nine, ten days or more), weeks (e.g. for one, two, three, four, five, six, seven, eight, nine, ten weeks or more), months (e.g. for one, two, three, four, five, six months or more), or years. If necessary, the composition may be administered for the duration of the life of the subject to be treated.

Alternatively, the composition may be administered to a subject only once during the life of said subject, or two or more times during the life of said subject on the same day or on different days separated by a period ranging for example from about 1 day to about 1 year, or more. The composition may thus be administered to a subject intermittently (e.g., every other day, every two days, or as necessary) or cyclically (e.g., administration for several days in a row, followed by an absence of administration, or “withdrawal,” for several days, at the end of which the cycle is repeated). In some cases, the composition may be administered periodically, for periods ranging for example from about 1 day to about 1 year, or more.

In some cases, the composition may be administered on demand (e.g., upon the appearance of symptoms of the disease associated with IL-23 expression, or prior to an increased risk of the manifestation of symptoms or the appearance of a disease associated with IL-23 expression).

Preferred doses include, but are not limited to, from 1 mg/kg to 10 mg/kg. In some embodiments, a dose is about 1 mg/kg, about 2 mg/kg, about 3 mg/kg, about 4 mg/kg, about 5 mg/kg, about 6 mg/kg, or about 7 mg/kg. In some cases, the dose is about 1 mg/kg/week, about 2 mg/kg/week, about 3 mg/kg/week, or about 4 mg/kg/week. In some cases, the dose ranges from about 1 mg/kg/week to about 15 mg/kg/week.

The duration and frequency of administration of the composition can be adapted by the skilled person, based on their general knowledge. In particular, the adaptation of these parameters will depend on the nature of the disease associated with IL-23 expression.

Of course, whatever dosage is used, it should be a safe and effective amount as determined by known methods, and as described herein. Moreover, the person skilled in the art can also determine, in light of their general knowledge, the effective amount of the composition that is to be administered to a human in order to achieve the desired biological effect.

The term “patient” or “subject” is used herein refers to any mammal, preferably any non-human animal or human individual, regardless of their age. The human individual may notably be an adult or child. The term “adult” refers herein to an individual of at least 16 years of age. The term “child” comprises infants from 0-1 years of age and children from 1-8 years of age, 8-12 years of age, and 12-16 years of age. The term “child” further comprises neonatal infants from birth to 28 days of age and post-neonatal infants from 28 to 364 days of age. The composition may be administered to an adult or a child, including neonatal infants.

### IV. Diseases Associated with IL-23 Expression

According to the present invention, the composition provided herein may be used in the prevention and/or treatment of a disease associated with IL-23 expression except cancers. To the inventors' knowledge, chalcones have never been shown to control IL-23 production. Such diseases may notably result from the expression of IL-23 itself and/or the expression of downstream cytokines regulated by IL-23, such as IL-17, and/or the activation of the IL-17/IL-23 pathway triggering the expression of combinations of molecules, such as combinations of pro-inflammatory molecules. The disease may be a chronic or acute disorder. The disease may be systemic or localized. The disease includes those pathological conditions that predispose a mammal to the disease in question. Such diseases may be present in humans and/or animals. As a non-limiting example, such diseases notably comprise autoimmune and/or inflammatory diseases, neurodegenerative diseases, non-infectious inflammatory respiratory diseases, immunological disorders, etc. A given disease may furthermore be classified in one or more categories according to its characteristics (e.g., a respiratory disease may also be considered to be an autoimmune disease and/or an inflammatory disease).

In the context of the present invention, the expression “a disease associated with IL-23 expression” as used herein is as defined above. In particular, it refers to a disease or disorder associated with an IL-23 expression that is pathogenic such as qualitatively and/or quantitatively different from the standard/expected one(s) in healthy subjects, yet in particular an IL-23 expression which is quantitatively increased or superior or excessive compared with the one of healthy subjects.

According to a further aspect of the present invention, said disease associated with IL-23 expression is preferably selected from an autoimmune and/or an inflammatory disease, a neurodegenerative disease, and a non-infectious inflammatory respiratory disease. In one aspect, said disease associated with IL-23 expression may be a disease other than cancers, in particular leukemia, and/or other than Alzheimer's disease. Thus, in a preferred aspect, the invention is directed to a composition comprising 2,4-D or a pharmaceutically acceptable salt thereof for use in the treatment of a disease associated with IL-23 expression, wherein said disease is selected from an autoimmune disease and/or an inflammatory disease, a neurodegenerative disease preferably other than Alzheimer's disease, and a non-infectious inflammatory respiratory disease. In another preferred aspect, the invention is directed to a composition comprising 2,4-D or a pharmaceutically acceptable salt thereof for use in the treatment of a disease associated with IL-23 expression, wherein said disease is selected from an autoimmune disease and/or an inflammatory disease, a neurodegenerative disease preferably other than Alzheimer's disease, and a non-infectious inflammatory respiratory disease. In another preferred aspect, the invention is directed to a composition comprising 2,4-D or a pharmaceutically acceptable salt thereof for use in the treatment of a disease associated with IL-23 expression, wherein said disease is selected from an autoimmune disease and/or an inflammatory disease, and a non-infectious inflammatory respiratory disease. In another preferred aspect, the invention is directed to a composition comprising 2,4-D or a pharmaceutically acceptable salt thereof for use in the treatment of a disease associated with IL-23 expression, wherein said disease is selected from an autoimmune disease and/or an inflammatory disease, and a non-infectious inflammatory respiratory disease.

The term “autoimmune disease” as used herein refers to a resulting from an immune response against a self-tissue or tissue component, including both self-antibody responses and cell-mediated responses. Indeed, it has been shown that IL-23 is associated with autoimmune disease and that targeting IL-23 improves autoimmune disease (see e.g., Tang et al., 2012). In one embodiment, “autoimmune disease” refers to a condition that results from, or is aggravated by, the production by B cells of antibodies that are reactive with normal body tissues and antigens. In other embodiments, the autoimmune disease is one that involves secretion of an autoantibody that is specific for an epitope from a self-antigen (e.g., a nuclear antigen). The term autoimmune disease, as used herein, encompasses organ-specific autoimmune diseases, in which an autoimmune response is directed against a single tissue, and non-organ specific autoimmune diseases, in which an autoimmune response is directed against a component present in several or many organs throughout the body. Autoimmune diseases notably include rheumatologic autoimmune diseases, gastrointestinal and liver autoimmune diseases, vasculitis, renal autoimmune diseases, dermatological autoimmune diseases, hematologic autoimmune diseases, atherosclerosis, uveitis, autoimmune ear diseases, Raynaud's syndrome, diseases associated with organ transplantation and autoimmune endocrine diseases such as diabetes.

Said autoimmune disease is preferably selected from rheumatologic autoimmune diseases, gastrointestinal and liver autoimmune diseases, vasculitis, renal autoimmune diseases, dermatological autoimmune diseases, hematologic autoimmune diseases, atherosclerosis, uveitis, autoimmune ear diseases, Raynaud's syndrome, diseases associated with organ transplantation and autoimmune endocrine diseases such as diabetes.

Rheumatologic autoimmune diseases comprise: rheumatoid arthritis such as acute arthritis, chronic rheumatoid arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, chronic progredien arthritis, arthritis deformans, chronic primary polyarthritis, reactive arthritis, and ankylosing spondylitis, Sjogren's syndrome, scleroderma, lupus such as SLE and lupus nephritis, polymyositis/cryoglobulinemia dermatomyositis, antiphospholipid antibody syndrome, and psoriatic arthritis. Preferably, said autoimmune disease is one or more of the above rheumatological diseases, more preferably rheumatoid arthritis. Indeed, IL-23 expression is associated with such diseases (see e.g., Dedong et al., 2019).

Gastrointestinal and liver autoimmune diseases comprise: autoimmune gastritis and pernicious anemia, autoimmune hepatitis, primary biliary cirrhosis, primary sclerosing cholangitis, and celiac disease. Preferably, said gastrointestinal and liver autoimmune disease is one or more of the above diseases. Indeed, IL-23 expression is associated with such diseases (see e.g., Aggeletopoulou et al., 2018).

Vasculitis comprises: ANCA—associated vasculitis, Churg-Strauss vasculitis, Wegener's granulomatosis, and polyarteritis. Preferably, said autoimmune disease is vasculitis. Indeed, IL-23 expression is associated with such diseases (see e.g., Nogueira et al., 2010).

Renal autoimmune diseases comprise: glomerulonephritis, syndrome Goodpasture, Berger's disease). Preferably, said renal autoimmune disease is one or more of the above diseases. Indeed, IL-23 expression is associated with such diseases (see e.g., Paust et al., 2009).

Dermatological autoimmune diseases comprise: psoriasis such as plaque psoriasis, guttate psoriasis, pustular psoriasis, and psoriasis of the nails, urticaria such as chronic allergic urticaria and chronic idiopathic urticaria, pemphigus vulgaris, bullous pemphigoid, lupus erythematosus, inflammatory hyperproliferative skin diseases, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, eczema including allergic or atopic eczema, asteatotic eczema, dyshidrotic eczema, and vesicular palmoplantar eczema. Preferably, said autoimmune disease is one or more of the above dermatological diseases, more preferably psoriasis. Indeed, IL-23 expression is associated with such diseases (see e.g., Di Meglio and Nestle, 2010).

Hematologic autoimmune diseases comprise thrombocytopenic purpura, thrombotic thrombocytopenic purpura, post-transfusion purpura, autoimmune hemolytic anemia. Preferably, said autoimmune disease is one or more of the above hematological diseases. Indeed, IL-23 expression is associated with such diseases (see e.g., Ye et al., 2015).

Autoimmune ear diseases comprise diseases such as inner ear disease and hearing loss. Preferably, said autoimmune disease is one or more of the above autoimmune ear diseases.

Autoimmune diseases associated with organ transplantation comprise graft rejection and Graft vs Host disease (GvHD). Preferably, said autoimmune disease is GvHD. Preferably, said organ transplant is selected from the group consisting of blood, bone marrow, stem cell, kidney, pancreas, liver, orthotopic liver, lung, heart, intestine, small intestine, large intestine, thymus, allograft stem cells, allograft of lesser intensity, bone, tendon, cornea, skin, cardiac valves, veins, arteries, blood vessels, stomach and testicle transplant. Indeed, IL-23 expression is associated with such diseases (see e.g., Das et al., 2009).

Autoimmune endocrine diseases comprise: juvenile onset (Type 1) diabetes mellitus, including pediatric insulin-dependent diabetes mellitus (IDDM), adult onset diabetes mellitus (Type II diabetes), autoimmune diabetes, idiopathic diabetes insipidus, and diseases related to diabetes (such as diabetic retinopathy, diabetic nephropathy, diabetic large-artery disorder) Addison's disease, autoimmune thyroid disease (such as Graves' disease, Hashimoto's thyroiditis, subacute thyroiditis, idiopathic hypothyroidism). Preferably, said autoimmune endocrine disease is diabetes, more preferably Type 1 diabetes. Indeed, IL-23 expression is associated with such diseases (see e.g., Zheng et al., 2018; Mensah-Brown et al., 2006).

Autoimmune diseases of the invention further comprise: atherosclerosis, uveitis, Raynaud's syndrome, etc. Indeed, IL-23 expression is associated with such diseases (see e.g., Pepple and Lin, 2018).

According to a preferred embodiment, said disease associated with IL-23 expression is selected from among the above autoimmune diseases. According to a particularly preferred embodiment, said autoimmune disease associated with IL-23 expression is selected from GvHD, psoriasis, and rheumatoid arthritis.

The term “inflammatory disease” as used herein refers to a disease, disorder, or condition characterized by having inflammation or an inflammatory component of body tissue. Inflammation may be localized or systemic. Inflammatory diseases notably include graft rejection including skin graft rejection; chronic inflammatory diseases of the joint including arthritis, rheumatoid arthritis, osteoarthritis, acute gouty arthritis, and inflammatory bone diseases (e.g., associated with increased bone resorption); inflammatory lung diseases such as asthma, adult respiratory distress syndrome, and chronic obstructive airway disease; Behcet's disease; inflammatory diseases of the eye including corneal dystrophy, trachoma, onchocerciasis, uveitis, sympathetic ophthalmitis and endophthalmitis; chronic inflammatory diseases of the gums including gingivitis and periodontitis; tuberculosis; leprosy; inflammatory diseases of the kidney including uremic complications, glomerulonephritis and nephrosis; inflammatory disorders of the skin including scleroderma, psoriasis and eczema; inflammatory diseases of the central nervous system, including chronic demyelinating diseases of the nervous system, infectious meningitis, encephalomyelitis, Parkinson's disease, Huntington's disease, amyotrophic lateral sclerosis and viral or autoimmune encephalitis; autoimmune disorders, immune-complex vasculitis; systemic lupus erythematosus (SLE); and inflammatory diseases of the heart such as cardiomyopathy, coronary thrombosis, ischemic heart disease, hypercholesterolemia, atherosclerosis); as well as various other diseases with significant inflammatory components, including preeclampsia, schizophrenia, chronic liver failure, brain and spinal cord trauma, or endometriosis. Indeed, IL-23 expression is associated with such diseases (see e.g., Debnath and Berk, 2017).

Preferably said inflammatory disease is selected from arthritis, rheumatoid arthritis, osteoarthritis, acute gout arthritis, an inflammatory bone disease; an inflammatory lung disease, preferably asthma, adult respiratory distress syndrome, or chronic obstructive airway disease; Behcet's disease; an inflammatory disease of the eye preferably corneal dystrophy, trachoma, onchocerciasis, uveitis, sympathetic ophthalmitis or endophthalmitis; a chronic inflammatory disease of the gums preferably gingivitis or periodontitis; tuberculosis; leprosy; an inflammatory disease of the kidney preferably a uremic complication, glomerulonephritis or nephrosis; an inflammatory disorder of the skin preferably psoriasis; a chronic demyelinating diseases of the nervous system; infectious meningitis; encephalomyelitis; Parkinson's disease; Huntington's disease; amyotrophic lateral sclerosis; an immune-complex vasculitis; systemic lupus erythematosus (SLE); an inflammatory disease of the heart, preferably cardiomyopathy, coronary thrombosis, ischemic heart disease, hypercholesterolemia, or atherosclerosis; preeclampsia; schizophrenia; chronic liver failure, or brain or spinal cord trauma; endometriosis. Preferably, said inflammatory skin disease is selected from among acne (e.g., acne vulgaris or acne conglobate), rosacea, psoriasis, eczema, atopic dermatitis, scleroderma, seborrheic dermatitis, boils, carbuncles, pemphigus, cellulitis, Grover's disease, hidradenitis suppurativa, lichen planus, or any other inflammatory skin disease described herein. Said inflammatory bone disease is preferably selected from among osteoporosis, periodontal disease, ankylosing spondylitis, osteoarthritis, Paget's disease, Lumbar disc herniation (LDH, including e.g., bulging disc, protruded disc, extruded disc, and sequestrated disc), or rheumatoid arthritis, or any other bone disease in which inflammation mediates bone loss or inflammatory bone disease described herein.

The term “neurological disease” as used herein refers to a central nervous system disorder. A “neurodegenerative disease” is a class of neurological disorder or disease, wherein the neurological disease is characterized by a gradual and progressive loss of neural tissue, and/or altered (e.g., reduced) neurological function. Typically reduced neurological function is a result of a gradual and progressive loss of neural tissue. Inflammatory responses in the central nervous system (CNS) have notably been observed in various chronic and acute neurodegenerative diseases, which are comprised in the scope of the present invention.

Said neurodegenerative disease may notably be selected from the group consisting of migraine headache, trigeminal neuralgia, myasthenia gravis, demyelinating diseases, such as acute transverse myelitis; extrapyramidal and cerebellar disorders, such as lesions of the corticospinal system; disorders of the basal ganglia; hyperkinetic movement disorders, such as Huntington's Chorea and senile chorea; drug-induced movement disorders, such as those induced by drugs which block CNS dopamine receptors; hypokinetic movement disorders, such as Parkinson's disease; progressive supranuclear Palsy; structural lesions of the cerebellum; spinocerebellar degenerations, such as spinal ataxia, Friedreich's ataxia, cerebellar cortical degenerations, multiple systems degenerations (Mencel, Dejerine-Thomas, Shi-Drager, and Machado-Joseph); systemic disorders (Refsum's disease, abetalipoproteinemia, ataxia, telangiectasia, and mitochondrial multi-system disorder); and disorders of the motor unit, such as neurogenic muscular atrophies (anterior horn cell degeneration, such as amyotrophic lateral sclerosis, infantile spinal muscular atrophy and juvenile spinal muscular atrophy); Down's Syndrome in middle age; Diffuse Lewy body disease; Senile Dementia of Lewy body type; Wernicke-Korsakoff syndrome; chronic alcoholism; Creutzfeldt-Jakob disease; Subacute sclerosing panencephalitis, Rasmussen's encephalitis, Huntington's disease, Hallerrorden-Spatz disease; Dementia pugilistica; neurotraumatic injury (e.g., spinal cord injury, brain injury, concussion, repetitive concussion); inflammatory pain; autism; depression; head trauma, stroke; cognitive disorders; epilepsy; macular degeneration and the like. Indeed, IL-23/IL-17 expression is associated with neurodegenerative diseases (see e.g., Fiala et al., 2010; Villegas et al., 2019; Li and Zhou et al., 2019).

Preferably, said neurodegenerative disease is selected from migraine headache, trigeminal neuralgia, myasthenia gravis, a demyelinating disease, preferably acute transverse myelitis; an extrapyramidal and cerebellar disorder; a hyperkinetic movement disorder, preferably Huntington's Chorea or senile chorea; a drug-induced movement disorder, preferably a disorder induced by drugs which block CNS dopamine receptors; a hypokinetic movement disorder, preferably Parkinson's disease; progressive supranuclear Palsy; a structural lesion of the cerebellum; a spinocerebellar degenerations, preferably spinal ataxia, Friedreich's ataxia, a cerebellar cortical degeneration, or a multiple systems degeneration; a systemic disorder, preferably Refsum's disease, abetalipoproteinemia, ataxia, telangiectasia, or mitochondrial multi-system disorder; a disorder of the motor unit, preferably a neurogenic muscular atrophy and more preferably amyotrophic lateral sclerosis; Down's Syndrome in middle age; Diffuse Lewy body disease; Senile Dementia of Lewy body type; Wernicke-Korsakoff syndrome; chronic alcoholism; Creutzfeldt-Jakob disease; Subacute sclerosing panencephalitis, Rasmussen's encephalitis, Huntington's disease, Hallerrorden-Spatz disease; Dementia pugilistica; neurotraumatic injury; inflammatory pain; autism; depression; head trauma, stroke; cognitive disorders; epilepsy; and macular degeneration. In a particular embodiment, said neurodegenerative disease associated with IL-23 expression is a disease other than Alzheimer's disease.

The term “non-infectious inflammatory respiratory disease” as used herein refers to any disease of the respiratory tract which is not due to a microbial infection. Said non-infectious respiratory disease may notably be selected from the group consisting of interstitial lung disease, cystic fibrosis, pulmonary oedema, chronic obstructive pulmonary disease, asthma, allergic alveolitis, restrictive lung disease (caused, for example, by work-related noxious substances, such as asbestosis or silicosis, or by lung tumors, such as lymphangiosis carcinomatosis, bronchoalveolar carcinoma and lymphoma), fibrosis, lupus erythematosus, systemic scleroderma or sarcoidosis, and rhinitis. Indeed, IL-23 expression in bronchial epithelial cells in various pathologies, including asthma and allergic sensitization has been described (Lee et al., 2017).

Preferably, said non-infectious respiratory disease is selected from the group consisting of interstitial lung disease, cystic fibrosis, pulmonary oedema, chronic obstructive pulmonary disease, asthma, allergic alveolitis, restrictive lung disease, fibrosis, lupus erythematosus, systemic scleroderma or sarcoidosis, and rhinitis.

According to a particularly preferred embodiment, said disease associated with IL-23 expression is selected from GvHD, psoriasis, rheumatoid arthritis, acute gout arthritis, amyotrophic lateral sclerosis, macular degeneration and endometriosis.

According to a more particularly preferred embodiment, said disease associated with IL-23 expression is selected from GvHD, psoriasis, rheumatoid arthritis and acute gout arthritis.

Advantageously, said disease associated with IL-23 expression is an inflammatory disease selected from the groups of auto-immune inflammatory diseases, such as rheumatoid arthritis, psoriasis, acute gout arthritis, periodontitis, uveitis, type-1 diabetes, autoimmune hepatitis, GvHD, and the like as mentioned above; of neurodegenerative inflammatory diseases, such as amyotrophic lateral sclerosis, macular degeneration, systemic lupus erythematosus, Parkinson's disease, Huntington's disease, and the like as mentioned above and of others IL-23-associated inflammatory diseases such as endometriosis, and the like as mentioned above).

The inflammatory disease associated with IL-23 expression may also be induced by viral infection, and in particular by chronic viral infection, except for the respiratory inflammatory disease. The expression “chronic viral infection” as used herein refers to a viral infection of humans or other animals in which the virus infects and replicates within the cells of said human or other animal over a prolonged period of time—usually weeks, months or years, without proving fatal. A chronically infecting virus may be a virus that is associated or correlated with the development of cancer. Chronic viral infection may ultimately lead to the induction of disease which may be fatal to the patient (e.g., as in the case of hepatitis C or hepatitis B virus-associated liver cancer or subacute sclerosing panencephalitis following reactivation of mutated measles virus in brain tissue). As a non-limiting example, said chronic viral infection may be due to one of the following viruses: herpes simplex virus (HSV) and other types of herpes virus, human papilloma viruses (HPV), hepatitis B virus (HBV), hepatitis C virus (HCV), Respiratory Syncytial Virus (RSV), influenza, Epstein Barr virus (EBV), Rubeola virus, Dengue virus (DEN), and the like. Indeed, IL-23/IL-17 expression is associated with chronic viral infections (see e.g., Jiang et al., 2018; Mebratu and Tesfaigzi, 2018; Wang et al., 2013; Uygun et al., 2019). The present invention is not only suitable for treating patients already infected with a chronic viral infection, but can also be applied prophylactically to prevent virus infection. This is especially recommended for subjects at risk of chronic viral infection.

According to a preferred embodiment, said chronic viral infection is selected from herpes viruses, such as HSV, HPV, HBV, HCV, RSV, influenza, EBV, Rubeola virus, and DEN, more preferably from among HSV, HBV, and HCV even more preferably from among HBV and HCV.

In some cases, a given subject may be at risk and/or diagnosed with two, three, four or more diseases associated with IL-23 expression that may be prevented and/or treated by the composition of the invention.

### V. Methods

According to another aspect, the present invention relates to a method of preventing and/or treating a disease associated with IL-23 expression, except cancers, in a subject in need thereof, comprising administering a therapeutically effective dose of the composition according to the invention to said subject. Preferably, said subject is a human subject. All aspects of the composition as described herein are comprised in the method of preventing and/or treating a disease associated with IL-23 expression. In particular, said composition comprises 2,4-D or a pharmaceutically acceptable salt thereof. Optionally, said composition may further comprise at least one additional therapeutic agent and/or at least one pharmaceutically acceptable carrier as provided herein. In some cases, said composition may comprise 2,4-D or a pharmaceutically acceptable salt thereof as the sole therapeutic agent. Said disease associated with IL-23 expression preferably comprises one or more of those provided herein.

Said prevention and/or treatment is(are) may be achieved via local, enteral or parenteral administration as provided herein. Preferably, said prevention and/or treatment is(are) may be achieved via a topical, oral, intraperitoneal, intravenous, intradermal, subcutaneous, or intraarticular route as provided herein. In cases where the method comprises administering of the composition comprising 2,4-D or a pharmaceutically acceptable salt thereof and at least one additional therapeutic agent, the composition comprising 2,4-D and the at least one therapeutic agent may be administered either simultaneously, or separately, or sequentially. As an example, the composition comprising 2,4-D may be administered orally (e.g., as an oral drug or a food supplement), whereas the at least one therapeutic agent is administered topically. As an alternative example, the composition comprising 2,4-D may be administered topically (e.g., as a cream or gel), whereas the at least one therapeutic agent is administered orally. All aspects provided herein are comprised said method.

According to another aspect, the present invention comprises a method of inhibiting IL-23 expression in at least one cell, comprising contacting said at least one cell with a composition comprising of the composition comprising 2,4-D or a pharmaceutically acceptable salt thereof. Preferably, said at least one cell is in a subject. Preferably said subject is a non-human animal or a human subject.

According to another aspect, a method of decreasing the activity of T-lymphocytes in a subject comprising administering of the composition comprising 2,4-D to said subject is provided herein, wherein the activity of T-lymphocytes in said subject is decreased. According to another aspect, a method of decreasing the proliferation of T-lymphocytes in a subject comprising administering of the composition comprising 2,4-D to said subject is provided herein, wherein the proliferation of T-lymphocytes in the subject is decreased. Preferably, said T-lymphocytes are Th17 lymphocytes.

### VI. Uses

According to another aspect, the present invention relates to the use of a composition comprising 2,4-D or a pharmaceutically acceptable salt thereof for the manufacture of a medicament for the prevention and/or treatment of a disease associated with IL-23 expression, except cancers. According to a further aspect, the present invention relates to the use of a composition comprising 2,4-D or a pharmaceutically acceptable salt thereof for the manufacture of a medicament intended for inhibiting or down-regulating IL-23 expression in a subject comprising administering to said subject so as to down-regulate IL-23 expression. All aspects as provided herein are comprised said use.

According to another aspect, the present invention relates to the use of the composition comprising 2,4-D or a pharmaceutically acceptable salt thereof for preventing and/or treating a disease associated with IL-23 expression, except cancers. All aspects as provided herein are comprised in said use. Preferably, said use is for preventing and/or treating a subject having an autoimmune, degenerative and/or inflammatory disease as described herein.

### VII. Kit

In another aspect, a kit containing materials useful for the treatment of the diseases described above is provided herein. The kit may comprise at least one container. Specifically, a pharmaceutical kit for preventing and/or treating a disease associated with IL-23 expression in a human in need thereof is provided herein, said kit comprising:
in a first container, a composition comprising 2,4-D as defined above;
optionally, in a second container, at least one therapeutic agent as defined above, wherein said composition comprising 2,4-D and, optionally, said at least one therapeutic agent, are in pharmaceutically acceptable carriers for administration according to any of the aspects provided herein.

In another aspect, a pharmaceutical kit is provided comprising:
a composition comprising 2,4-D as defined above; and
at least one therapeutic agent as defined above, as a combined pharmaceutical composition for simultaneous, separate or sequential use in preventing and/or treating a disease associated with IL-23 expression in a human in need thereof.

Suitable containers include, for example, bottles, vials, syringes, and test tubes. The containers may be formed from a variety of materials such as glass or plastic. Preferably, the first container comprises a composition comprising a therapeutically effective amount of 2,4-D. The container holds a composition that is effective for treating the condition and may have a sterile access port. For example, the container may be an intravenous solution bag or a vial having a stopper pierceable by a hypodermic injection needle. Alternatively, the container may be a tube or spray. A label may furthermore be located on or associated with the container which indicates that the composition is used for treating a given disease. The kit may further comprise a second container comprising a pharmaceutically-acceptable buffer, such as phosphate-buffered saline, Ringer's solution, and dextrose solution and/or at least one additional therapeutic agent. Said kit may further include other materials that are desirable from a commercial and user standpoint, such as other buffers, diluents, filters, needles, syringes, and instructions for use.

### EXAMPLES

The following examples are included to demonstrate preferred embodiments of the invention. All subject-matter set forth or shown in the following examples and accompanying drawings is to be interpreted as illustrative and not in a limiting sense. The following examples include any alternatives, equivalents, and modifications that may be determined by a person skilled in the art.

As illustrated in the examples below, IL-23 associated diseases can be prevented and/or treated by using the compositions comprising 2,4-D of the present invention. Such compositions are formulated for appropriate delivery. They are useful for preventing and/or treating both acute and chronic IL-23 associated diseases in mammals, more particularly in humans.

### Example 1. 2,4-D Inhibits IL-23 Expression

Monocytes and macrophages are an important part of the innate arm of the immune system. These cells are involved in inflammatory processes, with a profound capacity to synthesize and secrete pro- and anti-inflammatory cytokines. THP-1 cells, a human leukemia monocytic cell line, have been widely used as a model to study the immune response capacity of monocytes and monocyte-derived macrophages. More specifically, LPS-stimulated THP-1 monocytes and macrophages are widely used as a tool to study inflammation modulating effects of compounds (Chanput et al., 2010).

### Materials & Methods

2,4-D (>98% purity) was purchased from Sigma and dissolved in water for in vivo (intragastric administration) studies and in BPS for in vitro testing immediately before use.

The THP-1 cell line (TIB-202) was obtained from the American Type Culture Collection (ATCC, USA), grown in RPMI 1640 culture medium (Gibco) supplemented with 10% fetal bovine serum (Gibco, UK), 50 U/mL penicillin-streptomycin (Gibco), and 0.05 mM 2-Mercaptoethanol (Gibco). Cultured cells were plated into culture plates in appropriate volumes with a cell density of 500,000 cells/mL.

At two hours after stimulation with LPS (10 ng/ml) in the presence or not of 2,4-D at increasing concentrations, THP-1 cells from each experimental condition were directly lysed using TRIzol reagent (Invitrogen, USA) followed by RNA extraction according to the manufacturer's instructions. Finally, RNA was dissolved in 10 μL diethyl pyrocarbonate (DEPC)-treated water, quantified on a NanoDrop 2000 spectrophotometer (Thermo Scientific Inc., USA), and reverse-transcribed using the Super Script III First-Strand Synthesis System for RT-PCR kit (Thermo Scientific, USA) according to the manufacturer's instructions.

PCR was performed in duplicate using SYBR Green qPCR master mix (Takara Biomedical Technology Co., Ltd, Dalian, China) on an ABI 7500 Real-Time PCR System (Thermo, USA). The cycling program was as follows: denaturation at 95° C. for 15 s, 40 cycles of 95° C. for 15 s and extension at 60° C. for 60 s, followed by melt curve generation. The ΔΔCT method was used to quantify relative mRNA levels as described in User Bulletin #2 for the ABI PRISM 7700 Sequence Detection System (Applied Biosystems, 2001). The primer sequences used in this study are 5′-GCAGATTCCAAGCCTCAGTC-3′ (forward), 5′-TTCAACATATGCAGGTCCCA-3′ (reverse) for human IL-23p19 (SEQ ID NOs: 4 and 5, respectively); and 5′-ACAGTCCATGCCATCACTG-3′ (forward) and 5′-AGTAGAGGCAGGGATGATG-3′ for human GAPDH as a reference (SEQ ID NOs: 6 and 7, respectively). 24 hours after culture and treatments, supernatants were collected and IL-23 levels measured using the IL-23 Human Uncoated ELISA Kit (ThermoFisher Scientific Inc., USA) according to the manufacturer's instructions. A Varioskan LUX microplate reader (Thermo Fisher, USA) was used for the assay and 4-parameter logistic regression was used for data analysis. Two duplicates per sample were performed and results were averaged.

### Results

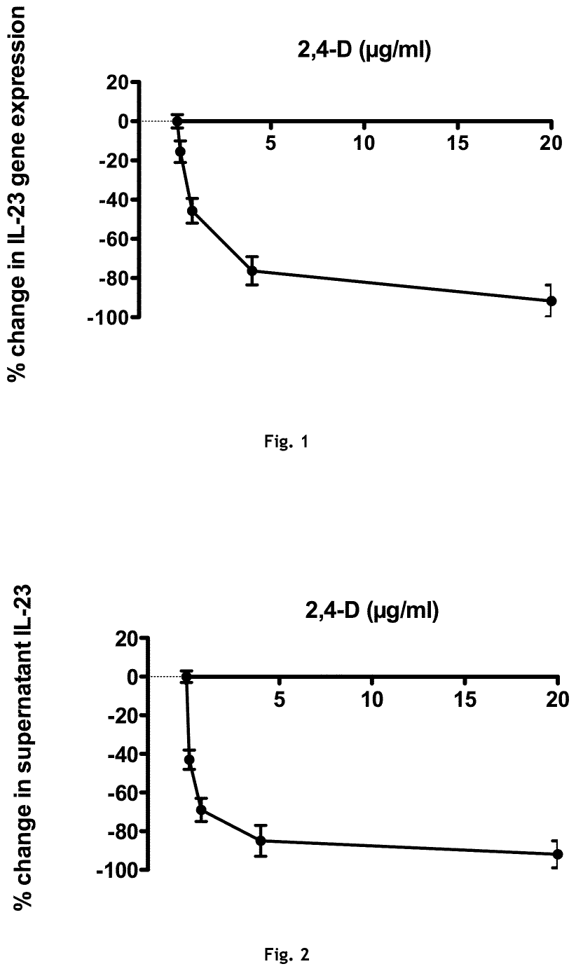

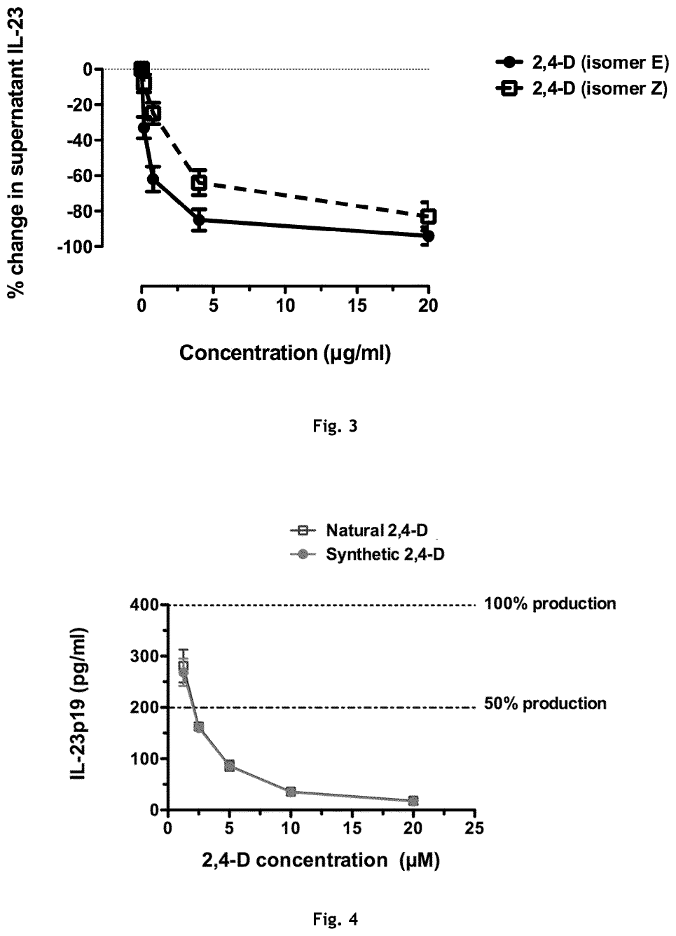

As shown in FIG. 1, 2,4-D strongly inhibited IL-23 gene expression in the THP-1 cell line expressing the IL-23 gene. IL-23 cytokine production by this cell line was also inhibited (see FIG. 2). Inhibition of IL-23 was dose-dependent, with maximum inhibition of IL-23 cytokine production observed at 20 μg/mL of 2,4-D in the LPS-stimulated THP-1 cell culture (FIG. 2). Advantageously, IL-23 detected in culture supernatant decreased by more than 90%, indicating that 2,4-D is a potent inhibitor of IL-23. Surprisingly, inhibition of IL-23 in the LPS-stimulated THP-1 cell culture was greater with the (E) isomer of 2,4-D than with the (Z) isomer (FIG. 3).

### Example 2. IC₅₀ of 2,4-d and Other Molecules in Inhibiting IL-23 Production in LPS-Stimulated THP-1 Cells

The effect of 2,4-D (in natural or synthetic forms) on IL-23 production was evaluated, in addition to a panel of additional molecules, including other chalcones.

### Materials & Methods

Molecules tested. Natural 2,4-D, isolated and purified from Chloranthus spicatus (purity HPLC≥98%, Cat. No.: 129742-43-2), was purchased from BOC Science (Shirley, NY). Synthetic 2,4-D (purity HPLC≥98%) was obtained from YIWEI Biotech (Shanghai, China). 2′-hydroxychalcone (2′-HC, purity HPLC≥98%, Cat. No.: 42224-53-3), 4-hydroxychlcone (4-HC, purity HPLC≥98%, Cat. No.: 20426-12-4), 2′,2-dihydroxychalcone (2′,2-DHC, purity HPLC≥98%, Cat. No.: IDF00045_ALDRICH), 2′,3-dihydroxychalcone (2′,3-DHC, purity HPLC≥98%, Cat No.: IDF00047_ALDRICH), 2′,4-dihydroxychalcone (2′,4-DHC, purity HPLC≥98%, Cat. No.: PH011685_ALDRICH), 2′,5′-dihydroxychalcone (2′,5′-DHC, purity HPLC≥98%, No.: IDF00046-1G), and 2′,4′,4-trihydroxychalcone (2′,4′4-THC, purity HPLC≥98%, No.: 38986_SIAL) were purchased from Sigma. 2′,4′-dihydroxychalcone (2′,4′-DHC, purity HPLC≥98%, Cat. No. AB151736) was purchased from abcr GmbH (Karlsruhe, Germany). Isobavachalcone (IBC), 4′-hydroxy-2′6′-dimethoxychalcone (HMC), 2-trifluoromethyl-2′-methoxychalcone (TFM) and Hydroxysafflor yellow A (HYA) were also tested.

Cell culture. THP-1 cells were cultured in RPMI 1640 culture medium (Gibco) supplemented with 10% fetal bovine serum (FBS; Gibco, UK.), 0.05 mM 2-mercaptoethanol, and 1% penicillin/streptomycin. Cells were used in exponential growth phase, with culture medium being replaced with fresh medium 1 day prior to the experiment. The following day, THP-1 cells were plated into 24-well culture plates with a cell density about 800,000 cells/mL and 2 mL per well. 10 ng/mL LPS was used to stimulate the THP-1 cells. RPMI 1640 medium containing 10% FBS and 1% P/S was used as a normal control (NC). 10 mM stock solutions of compounds in DMSO were diluted to 20 μM with complete culture medium just before use. Experiments were performed in quadruplicate.

Enzyme linked immune-sorbent assay (ELISA). At 24 hours, after centrifugation at 300 g for 10 min., 0.5 mL of supernatant was collected and stored at −80° C. until use. IL-23 levels were determined in culture supernatants as described in Example 1.

### Results

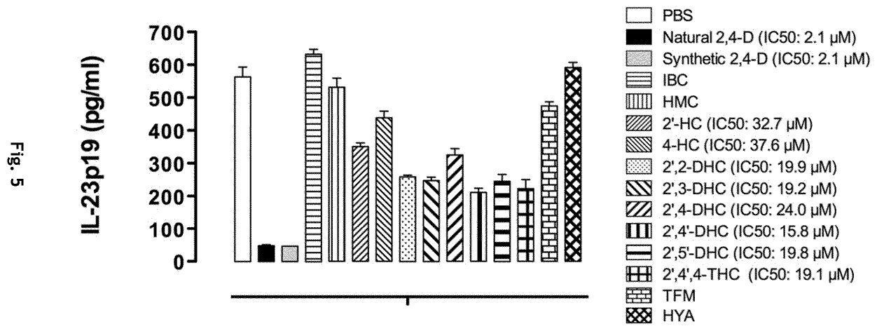

Both naturally isolated and chemically synthesized 2,4-D showed similar inhibition of IL-23, with an IC50 of approximately 2.1 μM (FIG. 4).

In contrast to 2,4-D, IBC, HMC, and HYA do not inhibit IL-23 production. Remaining compounds show inhibition of IL-23; however, 2,4-D unexpectedly shows inhibition of IL-23 at levels that are ˜7,5- to 18-fold superior to remaining compounds (FIG. 5). Thus, 2,4-D has an unexpected effect on IL-23 that is furthermore superior to other compounds, including other chalcones.

### Example 3. 2,4-D Successfully Treats Rheumatoid Arthritis in the Mouse Model

The expression of the IL-23 and IL-17 genes and the production of their cognate cytokines, IL-17 and IL-23, are known to be important in the pathogenesis of rheumatoid arthritis. Furthermore, it has been shown the administration of an anti-mouse IL-17 antibody reduces symptoms in a mouse collagen induced arthritis model as compared to control mice (Bai et al., 2014). A deficiency of IL-23/p19 has also been shown to be protective in this same model (Murphy et al, 2003), thus illustrating that IL-23 antagonists may be beneficial in treating this disease.

The effect of 2,4-D in the mouse model of rheumatoid arthritis was evaluated here.

### Materials & Methods

2,4-D. 2,4-D (>98% purity) was purchased from Sigma and dissolved in water for in vivo (intragastric administration) studies immediately before use.

Induction of collagen induced arthritis (CIA) in DBA/1 mice treated with oral 2,4-D. Forty DBA/1 mice (8-12 weeks of age; 20 males and 20 females) were obtained from the Center for Experimental Animals in Beijing, China. The CIA model was established according to the standard Hooke Protocol (accessible at https://hookelabs.com/protocols/cialnduction_DBA1.html). Complete Freund's adjuvant (CFA) and incomplete Freund's adjuvant (IFA) were purchased from Hooke Laboratories. 35 days after immunization, the 40 immunized mice were distributed randomly into 4 groups (10 controls, 10 treated with 0.2 mg daily oral 2,4-D, 10 treated with 1 mg daily oral 2,4-D for 15 days). All animals were followed for an additional 15 days post-treatment.

CIA scoring. CIA is scored on a scale of 0 to 16 (0 to 4 for each paw, adding the scores for all 4 paws), using the following criteria:

TABLE 1
CIA scoring

| Score | Clinical observations |
|---|---|
| 0 | Normal paw. |
| 1 | One toe inflamed and swollen. |
| 2 | More than one toe, but not the entire pawn inflamed and swollen, |
| | or mild swelling of entire paw. |
| 3 | Entire paw inflamed and swollen. |
| 4 | Very inflamed and swollen paw or ankylosed paw. If the paw is |
| | ankylosed, the mouse cannot grip the wire top of the cage. |

Statistical analysis. Data are presented as the mean±SEM for the in vivo animal study. Statistical analysis was performed using Student's t-test (paired 2-sample testing).

### Results

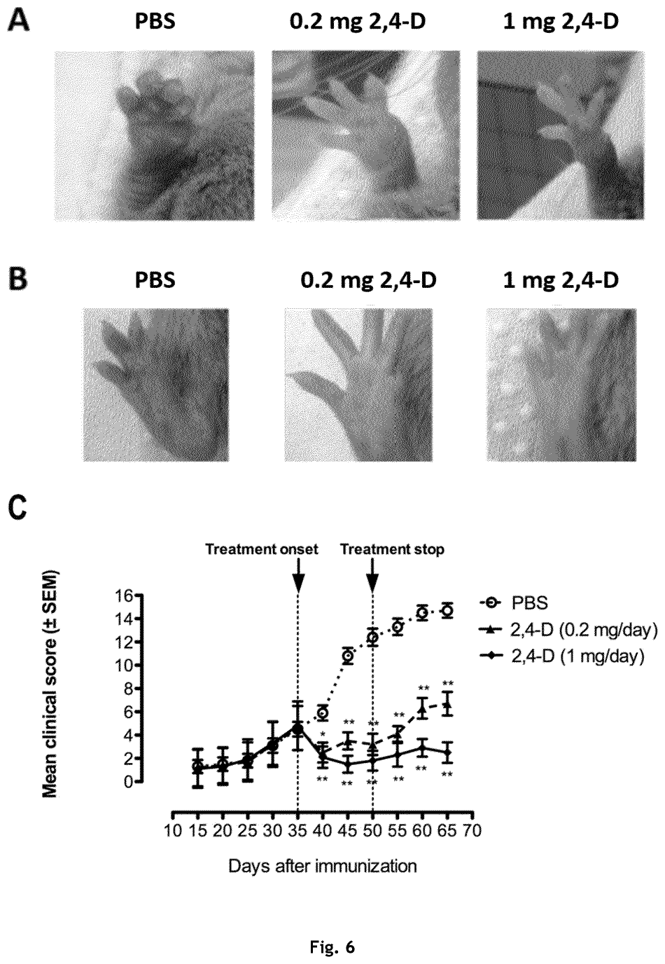

The mean clinical score (±SEM) was strongly decreased in CFA/IFA-immunized mice treated with either high (1 mg per day) or low doses (0.2 mg per day) of 2,4-D as from day 5 post-treatment (2- to 7-fold and 2- to 3-fold decreases when compared with the control group for the high and low dose, respectively, p<0.01). This suppression of CIA was maintained in CFA/IFA-immunized mice treated with a high dose of 2,4-D even 15 days after withdrawal of treatment (FIG. 6).

### Example 4. 2,4-D Inhibits Expression of IL-23 and IL-17 In Vitro in RA-Patient Synovial Cells in a Concentration-Dependent Manner

### Materials & Methods

Synovial fluid was freshly collected from 20 patients with rheumatoid arthritis (RA) by arthrocentesis. At two hours after treatment or not with 2,4-D, RA-patient synovial cells were directly lysed using TRIzol reagent (Invitrogen, USA) followed by RNA extraction according to the manufacturer's protocol. Finally, RNA was dissolved in 10 μL DEPC-treated water, quantified on a NanoDrop 2000 spectrophotometer (Thermo Scientific Inc., USA), and reverse-transcribed using the Super Script III First-Strand Synthesis System for RT-PCR kit (Thermo Scientific, USA) according to the manufacturer's protocol. PCR was performed in duplicate using SYBR Green qPCR master mix (Takara Biomedical Technology Co., Ltd, Dalian, China) on an ABI 7500 Real-Time PCR System (Thermo, USA). The cycling program was as follows: denaturation at 95° C. for 15 s, 40 cycles of 95° C. for 15 s and extension at 60° C. for 60 s, followed by melt curve generation. The ΔΔCT method was used to quantify relative mRNA levels as described in Example 1 above. The primer sequences specific for human IL-17 genes (hu-IL-17 Forward: 5′-CAACCGATCCACCTCACCTT-3′; hu-IL-17 Reverse: 5′-GGCACTTTGCCTCCCAGAT-3′) (SEQ ID NOs 8 and 9, respectively) and IL-23 (see Example 1, above) were used.

### Results

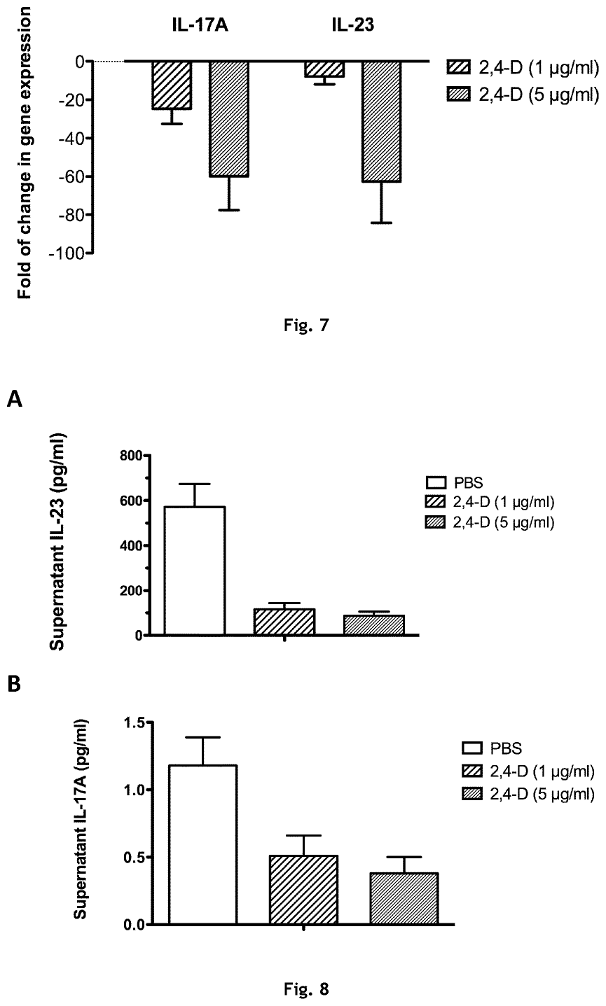

As shown in FIG. 7, the expression of IL-17 and IL-23 in synovial fluid cell culture was decreased 23-fold and 8-fold, respectively, when treated with 1 μg/ml of 2,4-D (p<0.05). This inhibition of IL-17 and IL-23 expression reached as high as 60-fold and 65-fold, respectively, when RA patient synovial cells were treated with 5 μg/ml of 2,4-D (p<0.01). Similarly, the concentrations of IL-17 and IL-23 protein in the culture supernatant were significantly inhibited by the presence of 2,4-D (FIG. 8, A, B).

### Example 5. 2,4-D Successfully Treats Psoriasis in the Mouse Model

Psoriasis, is the most frequent human auto-immune disease, affecting an estimated 2-4% of the population. It has been shown that IL-23 is functionally involved in the pathogenesis of psoriasis. It has also been observed that the expression of IL-23 is increased in psoriatic skin lesions where increased numbers of Th17 cells are present. Furthermore, monoclonal antibodies targeting IL-17A, such as secukinumab, have been approved by health authorities for use in the treatment of psoriasis (see e.g., Roman et al., 2015). An anti-IL-23 antibody, guselkumab, has also been approved for the treatment of plaque psoriasis (Yang et al. 2019).

Here, we evaluated the anti-psoriasis effect of 2,4-D using the 12-O-Tetradecanoyl Phorbol-13-Acetate (TPA)-induced psoriasis mouse model.

### Materials & Methods

2,4-D. 2,4-D (>98% purity) was purchased from Sigma and dissolved in water for in vivo (intragastric administration) studies immediately before use.

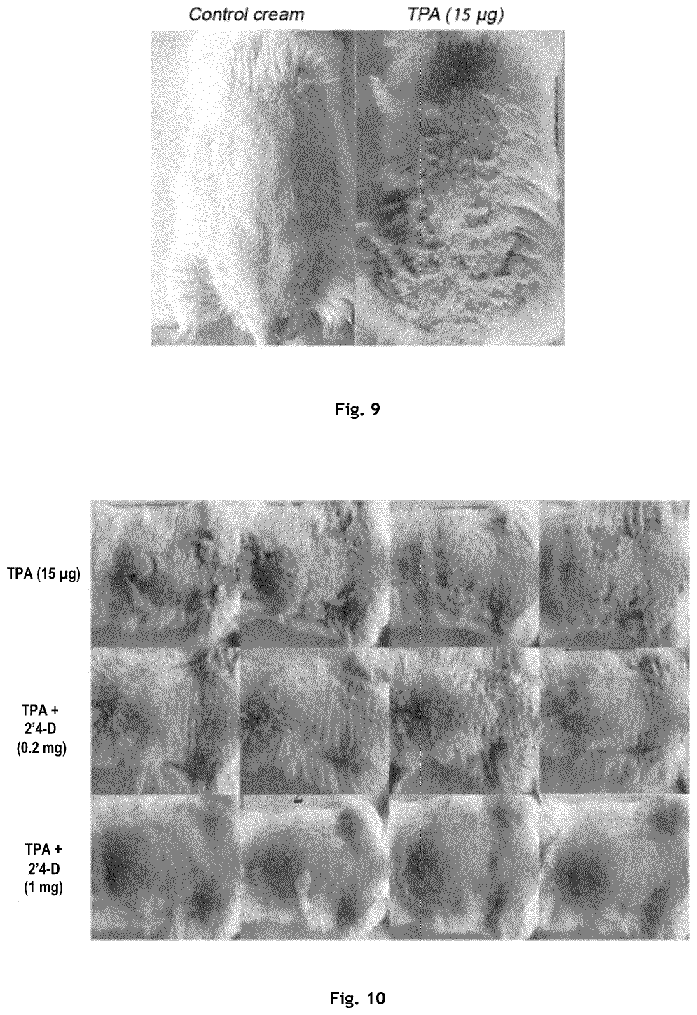

Induction of TPA-induced psoriasis-like skin hyperplasia in mice treated with oral 2,4-D. Forty mice (BALB/c) were purchased from Guangdong Experimental Animal Center. All experiments were approved by the animal ethics committee of Guangzhou University of Chinese Medicine according to Chinese legislation on animal experiments. Mice (n=30) at 8 to 11 weeks of age received a daily topical dose of 15 μg TPA (Sigma) in 200 μl of acetone on the shaved back for day 1 and 4. This dose was empirically determined to cause the most optimal and reproducible skin inflammation in mice (data not shown). Control mice (n=10) were treated similarly with a control vehicle cream (Sigma). See FIG. 9 for an illustrative example of a mouse receiving topical TPA as compared to a control mouse receiving a control vehicle cream. Of the 30 mice that received topical TPA, 20 mice were treated orally with 0.2 mg (n=10) or 1 mg (n=10) 2,4-D daily until day 6. The remaining 10 untreated TPA mice served as a disease control. On day 7, dorsal skin was photographed and fixed in 10% formalin and stained with H&E (Haemotoxylin and Eosin). Epidermal thickness was quantified by Image J software.

Statistical analysis. Data are presented as the mean±SD for the in vivo animal study. Statistical analysis was performed using Student's t-test (paired 2-sample testing).

### Results

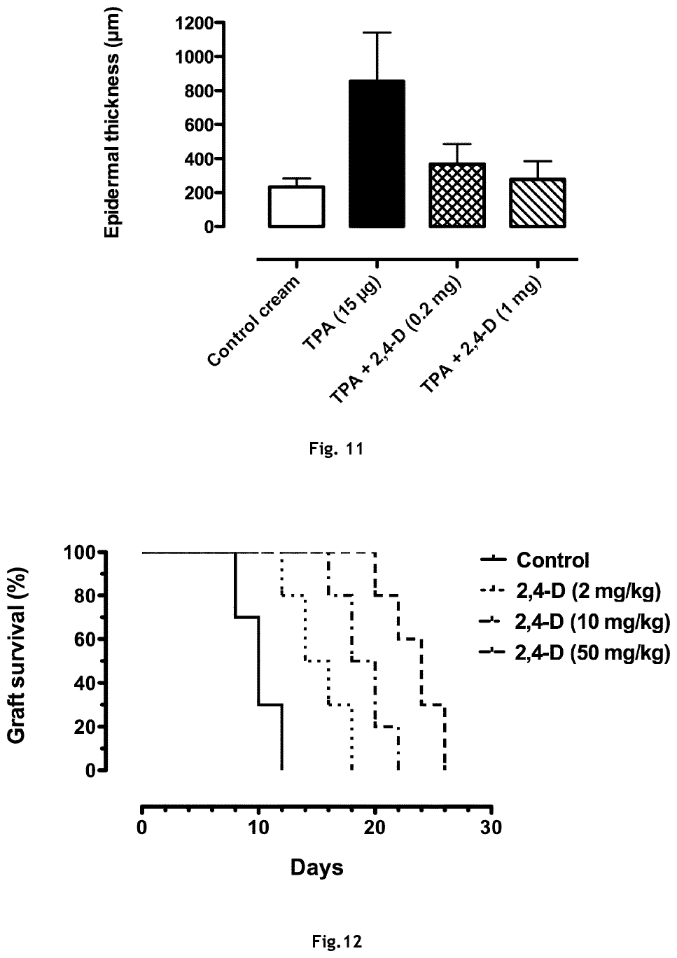

Dorsal skin of mice receiving a daily dose of TPA and, optionally, 0.2 or 1 mg of 2,4-D are shown in FIG. 10. Dorsal skin of mice treated with 0.2 or 1 mg 2,4-D is improved in a dose-dependent manner when compared to mice receiving TPA only. The thickness of TPA-induced psoriasis-like back skin hyperplasia was significantly lower in mice treated with 0.2 mg (n=10; p<0.05) or 1 mg (n=10; p<0.01) of daily oral 2,4-D as compared with untreated mice. Although the thickness of back skin in TPA mice treated with 0.2 mg of 2,4-D was significantly higher than those normal skin observed in mice treated only with control cream (p>0.05), the thickness of back skin in TPA mice treated with 1 mg of 2,4-D was similar to the thickness of normal skin observed in control mice (p>0.05) (FIG. 11).

### Example 6. 2,4-D Successfully Prevents Graft Rejection

Graft versus host disease (GVHD) is the major complication associated with allogeneic cell and tissue transplantation. IL-23 secretion has been identified as a key factor in organ-specific pathogenesis (Das et al., 2009). It has been furthermore been shown that anti-IL-12/23 p40 antibodies attenuate experimental chronic graft-versus-host disease (Okamoto et al., 2015).

Here, we evaluated the effect of 2,4-D on graft rejection in the mouse model.

### Materials & Methods

2,4-D. 2,4-D (>98% purity) was purchased from Sigma and dissolved in water for in vivo (intragastric administration) studies immediately before use.

Skin transplantation. Murine full-thickness allo-skin transplantation to study graft rejection was conducted according to a previously well-described protocol (Cheng et al, 2017). All procedures were conducted in accordance with the Guide for the Care and Use of Laboratory Animals of the National Institute of Health (NIH) and were approved by the animal ethics committee of Guangzhou University of Chinese Medicine.

Briefly, donor mice (Balb/c) were anesthetized with isoflurane (induction vaporizer at 4%, maintenance at 1-2% through the mouse cone). The toe pinch withdrawal reflex was used to monitor the depth of anesthesia. The back of the animal was shaved with an electric razor and disinfected with 10% povidone iodine. Using sterile scissors, gloves, and aseptic technique, donor back skin was harvested from the hip to the neck with blunt dissection at the level of the areolar connective tissue. After harvesting the skin graft, the animal was euthanized by cervical dislocation. Under a microscope, the connective tissue, fat tissue, and panniculus carnosus were separated from the back skin using fine tenotomy scissors. Using sterile technique, 15-mm×15-mm grafts were cut out from the back skin for a 10-mm×10-mm to 15-mm×15-mm graft bed (10 grafts were obtained from one donor mouse). The grafts were stored on gauze soaked with sterile phosphate-buffered saline (PBS) in a petri dish on ice.

The recipient mice (C57BL/6) were anesthetized with isoflurane. Buprenorphine (0.02 mg/kg) was administrated for postoperative pain relief. The side of the back of the animal where the graft will be inserted was shaved and disinfected with 10% povidone iodine. A 10-mm×10-mm to 15-mm×15-mm square of skin was intersected using scissors. The defect size was slightly larger (10%) than the graft. The graft was positioned on the graft bed avoiding folds along the edges. Eight sutures were placed on the corners and on the middle of each edge. For each suture, the needle was passed through the graft and then through the panniculus carnosus of the graft bed below the surrounding recipient skin. The anesthetic mask was then removed to let the animal recover partially from anesthesia before applying an adhesive bandage. The recipient mouse was wrapped in the adhesive bandage with folded gauze over the graft. The bandage was made by combining two bandages, cutting the adhesive part of one and placing the two absorbent pads together. Enrofloxacin (5 mg/kg) was administered after surgery for infection prophylaxis. The transplanted mouse was placed in a clean cage over a microwavable heating pad until it fully recovered from anesthesia. After 1 hour of close postoperative observation, the mouse was returned to the housing facility. Seven days after surgery, the mouse was anesthetized as indicated above and the bandage was removed by cutting the ventral side of the bandage. The graft was observed and palpated on the following day for signs of scabbing, contraction, or hardness. If present, the graft may not have achieved proper vascularization and was considered as a technical failure. When the graft was considered as successful, it was then monitored daily for signs of rejection. Grafts were considered as rejected when ≥90% of the graft tissue became necrotic. Animals were euthanized at day 30 for tissue harvest.

Using this model of skin transplantation, we investigated a novel approach targeting IL-23/IL17 axis to prevent graft rejection. The Balb/c to C57BL/6 skin graft was orally administered (intragastric, bid) from the day of transplantation until rejection with 2 mg/kg (n=10), 10 mg/kg (n=10), and 50 mg/kg (n=10) of 2,4-D dissolved in 0.2 ml water. Ten grafts without treatment served as a control.

Statistical analysis. Data are presented as the mean±SD for the in vivo animal study. Statistical analysis was performed using Student's t-test (paired 2-sample testing).

### Results

In complete mismatch models (Balb/c to C57BL/6), full-thickness skin grafts are usually rejected in 8 to 14 days. In contrast, syngeneic full-thickness skin transplantations (Balb/c-Balb/c) consistently show no evidence of rejection until 30 days. Using this skin transplantation model, we investigated a novel approach of suppressing IL-23 gene expression by 2,4-D, susceptible to prevent graft rejection.

Using Balb/c as donors and C57BL/6 as recipients, we observed that inhibiting IL-23 gene expression with different doses of 2,4-D (2, 10, and 50 mg/kg; bid) resulted in a significant increase (p<0.001) of graft survival in a dose-dependent manner (median graft survival rates of 15, 19, 24 days for the dose group of 2, 10, and 50 mg/kg respectively, as compared to the control groups median graft survival of 10 days) (FIG. 12).

### Example 7. Anti-Inflammatory Effect of 2,4-D is Mediated Specifically Through the Down-Regulation of IL-23 Gene Expression

### Materials & Methods

Briefly, before the experiment, 48-well plates were coated with 150 μl/well hamster anti-mouse CD3 (Clone 145-2C11, NA/LE, BD PHM, USA) at a concentration of 10 μg/ml and incubated overnight at 4° C. The next day, a C57BL/6 mouse was euthanized by cervical dislocation, and its spleen was aseptically dissected using sterile techniques. The harvested spleen was placed in a small Petri dish containing 10 ml of ice-cold PBS/FBS, ground using 2 frosted microscope slides, and filtered through a piece of 40 μm nylon material to yield a single cell suspension. Thereafter, the spleen cell suspension was carefully dispensed onto mouse lymphocyte separation medium (TBD Science Inc, Tianjin, China) and the lymphocytes were collected after density gradient centrifugation. After washing and cell counting, cell density was adjusted to 1 million cells/mL with RPMI 1640 (Gibco) plus 10% FCS and 1 μg/mL anti-mouse CD28 (BD PHM). According to the experimental setting, test compounds at desired concentration (0.1% DMSO final concentration) or vesicle controls were added with or without recombinant mouse IL-23 (10 ng/mL, carrier-free, eBioscience, USA). After stimulation for 72 hours with anti-CD3 and anti-CD28 monoclonal antibodies, culture supernatants were collected and IL-17A measured with an IL-17A mouse ELISA kit (Thermo Fisher Scientific Inc.) according to the manufacturer's instructions. Cell count and viability were also evaluated with 0.4% trypan blue (Thermo Fisher Scientific Inc.) staining.

### Results

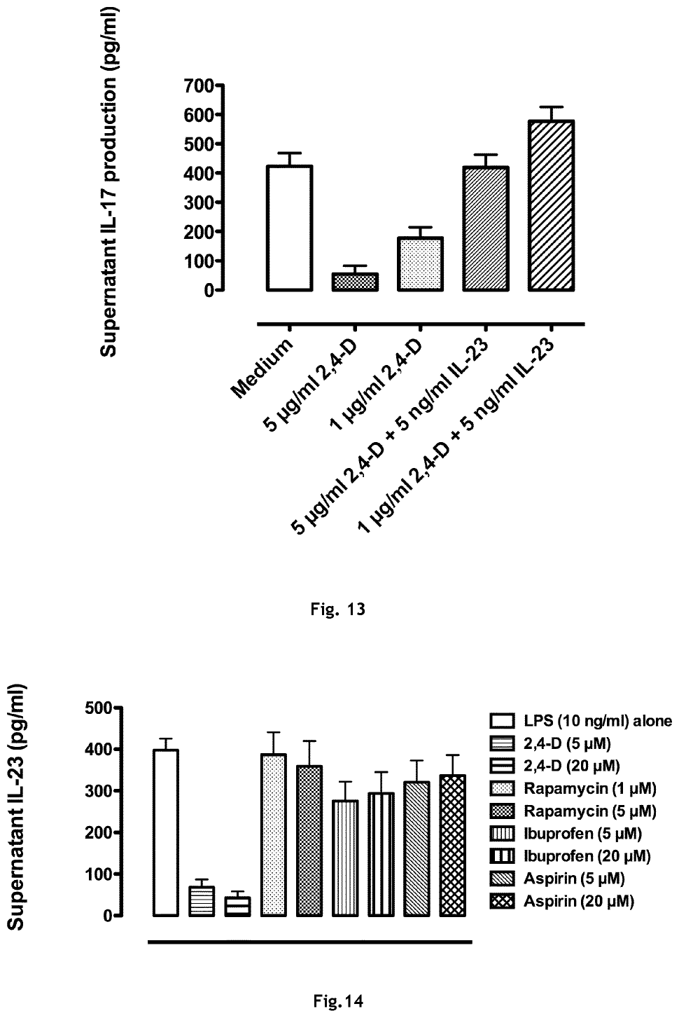

As shown in FIG. 13, anti-CD3/CD28 stimulated spleen cells produced and released as much as 420 μg/ml of supernatant IL-17. The production and release of IL-17 in supernatant was significantly suppressed (p<0.001) by both 1 μg/ml and 5 μg/ml of 2,4-D. However, 2,4-D-derived suppression was completely reversed by adding recombinant mouse IL-23 (5 ng/ml) (FIG. 13).

### Example 8. 2,4-D is a Potent Anti-IL-23 Agent as Compared to NSAIDs

### Materials & Methods

The anti-IL-23 activity of 2,4-D was compared with the commercially prescribed nonsteroidal anti-inflammatory drugs (NSAIDs:Rapamycin, Ibuprofen, and Aspirin) in the LPS-stimulated THP-1 cell culture system described above (Example 1)).

### Results

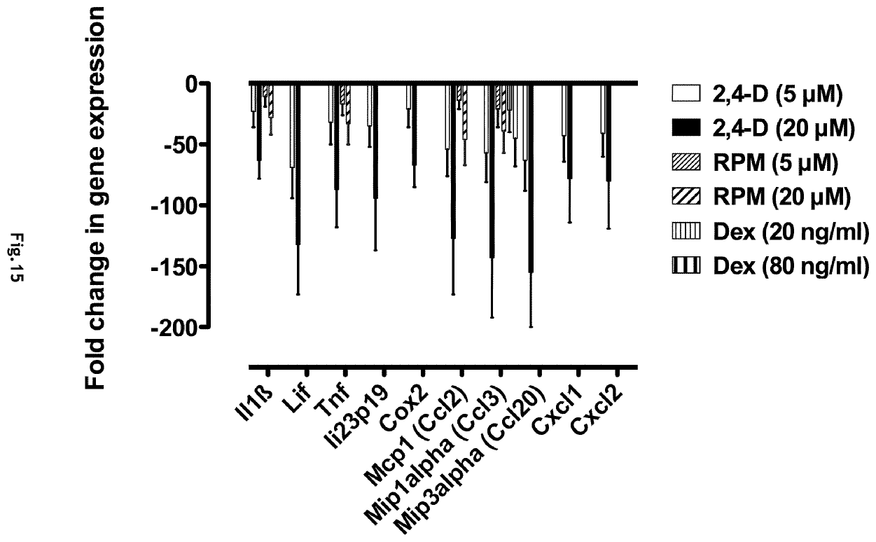

As shown in FIG. 14, 2,4-D led to a 5 to 10-fold inhibition in IL-23 production/release in supernatant (p<0.001), while Ibuprofen led to merely a 20-25% reduction of IL-23 release in supernatant (p<0.05). In contrast, no significant change of IL-23 production/release in supernatant was observed in the presence of Rapamycin or Aspirin (FIG. 14).

### Example 9. 2,4-D Suppresses Transcriptions of IL-17/IL-23 and TNF Gene Signatures as Well as Corresponding Protein Releases

### Materials & Methods

Sample Collection and Preparation. THP-1 cells, a human leukemia monocyte-derived cell line, have been widely used as a model to study the modulation of the immune response and the function of monocytes and monocyte-derived macrophages. Here, lipopolysaccharide (LPS)-stimulated THP-1 cells were used as a model to study the cytokine storm in vitro (Chanput et al., 2014). THP-1 cells (purchased from American Type Culture Collection, Rockville, MD) were grown in RPMI 1640 culture medium (Gibco) supplemented with 10% fetal bovine serum (FBS) (Gibco, UK), 0.05 mM 2-mercaptoethanol, and 1% penicillin/streptomycin (Invitrogen) at 37° C. in 5% CO₂ in a humidified incubator. Cultured cells were plated into culture plates in appropriate volumes with a cell density of 5×10⁵ cells/ml. 2,4-D was purchased from Wuhan ChemFaces Biochemical Co., Ltd (Hubei, China) and tested for cytotoxicity in THP-1 cells overnight using a commercial MTT Cell Proliferation and Cytotoxicity Assay Kit (Beyotime, Shanghai, China). THP-1 cells were treated with PBS alone as negative control (NTC), with 10 ng/mL LPS alone (Sigma, St. Louis, MO) as the model of cytokine storm in vitro (MODEL), or with 10 ng/mL LPS and 5 μM or 20 μM 2,4-D, 5 μM or 20 μM rapamycin (RPM) (Sigma), or 20ng/μL or 80 μg/mL dexamethasone (DEX) (Sigma). THP-1 cells were harvested at different time points ranging from 0.5-24 h to investigate gene expression kinetics, while cell-free culture supernatants were collected and stored at −80° C. to measure time-dependent cytokine secretion as described. Cell cultures were performed in quadruplicate wells for each experiment.

Real-Time Quantitative PCR. Real-time quantitative PCR was performed in triplicate using the TB Green qPCR master mix (Takara Biomedical Technology Co., Ltd, Dalian, China) on an ABI 7500 Real-Time PCR System (Thermo, USA). The cycling program was as follows: denaturation at 95° C. for 15 s, 40 cycles of 95° C. for 15 s, and extension at 60° C. for 60s, followed by generation of the melting curve. The ΔΔCT method was used to quantify relative mRNA levels as described above in Example 1 (Applied Biosystems). Data are expressed as mean±SD. The primer sequences specific for human genes which were used are listed in the following table.

TABLE 2

| SEQ ID | | | |
|---|---|---|---|
| NOS | Gene | Forward Primer (5′-3′) | Reverse Primer (5′-3′) |
| 10, 11 | IL-1ß | ACAGATGAAGTGCTCCTTCCA | GTCGGAGATTCGTAGCTGGAT |
| 12, 13 | LIF | CAGTGCCAATGCCCTCTTTAT | GGCCACATAGCTTGTCCAGG |
| 14, 15 | TNF-alpha | GGCTCCAGGCGGTGCTTGTTC | AGACGGCGATGCGGCTGATG |
| 4, 5 | IL-23p19 | GCAGATTCCAAGCCTCAGTC | TTCAACATATGCAGGTCCCA |
| 16, 17 | COX-2 | GAATCATTCACCAGGCAAATTG | TCTGTACTGCGGGTGGAACA |
| 18, 19 | MCP-1 | TCATAGCAGCCACCTTCATTC | TAGCGCAGATTCTTGGGTTG |
| | (CCL2) | | |
| 20, 21 | MIP-1 alpha | TTGTGATTGTTTGCTCTGAGAGTTC | CGGTCGTCACCAGACACACT |
| | (CCL3) | | |
| 22, 23 | MIP-3alpha | TTCACCCAAGTCTGTTTTGGA | GAAGGCTGTGACAATGCT |
| | (CCL20) | | |
| 24, 25 | CXCL1 | AGTGGCACTGCTGCTCCT | TGGATGTTCTTGGGGTGAAT |
| 26, 27 | CXCL2 | CTGCTCCTGCTCCTGGTG | AGGGTCTGCAAGCACTGG |
| 6, 7 | GAPDH | ACAGTCCATGCCATCACTG | AGTAGAGGCAGGGATGATG |

ELISA. THP-1 culture supernatants were collected at 24 hours. IL-23 Human Uncoated ELISA Kit (88-7237-86) was purchased from ThermoFisher (ThermoFisher, Vienna, Austria). Human IL-1β, IL-6, IL-10, TNF-α, MCP-1, and MIP-1alpha ELISA kits were purchased from Mabtech (Stockholm, Sweden). Culture supernatants were measured according to the manufacturer's instructions. A Varioskan LUX microplate reader (Thermo Fisher, USA) was used for the assay and 4-parameter logistic regression was used for data analysis. Supernatants were taken from quadruplicate wells for each sample and results were averaged. Data are expressed as mean±SD.

Statistical analysis. Statistical analysis was performed using Student's t-test (paired 2-sample testing), with * corresponding to a p-value<0.05 and ** corresponding to a p-value<0.01.

### Results

Expression levels of IL-17/IL-23 and TNF signaling pathway-associated cytokine genes (Tnf, IL1ß, Lif, IL23p19, Cox2) and chemokine genes (Ccl2/Mcp1, Ccl3/Mip1alpha, Ccl20/Mip3alpha, Cxcl1, and Cxcl2) were compared in the TPH-1 model treated for 2 h with different concentrations of 2,4-D (5-20 μM), RPM (5-20 μM), or DEX (20-80 ng/ml).

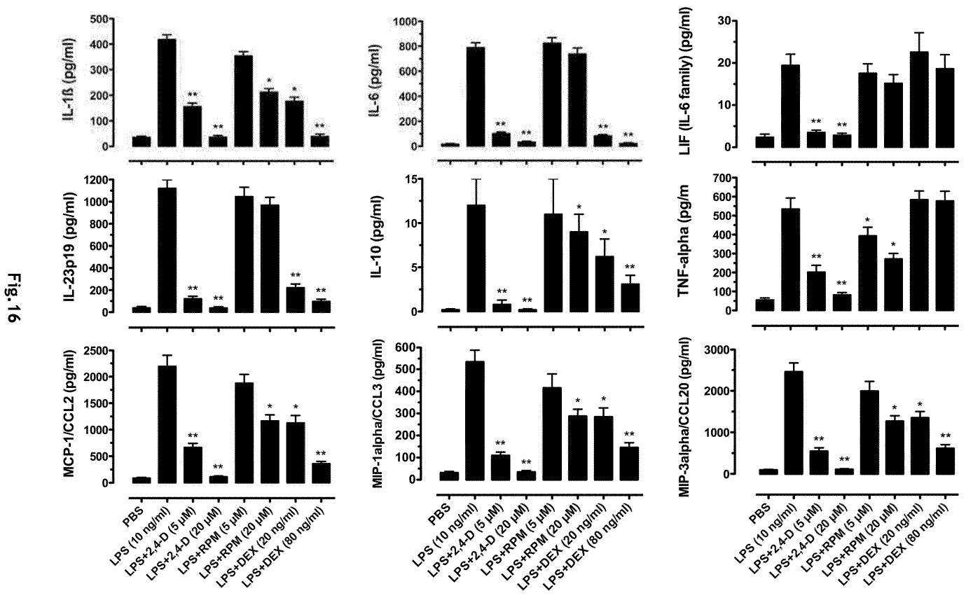

As shown in FIG. 15, 2,4-D suppressed transcription of IL-17/IL-23 and TNF signatures by 63-143-fold (p<0.01) in a dose-dependent manner while RPM slightly inhibited the transcription of Tnf, II1ß, Ccl2, and Ccl3 (i.e., by ≤50-fold; p<0.01), but not Lif, Ii23p19, Cox2, Ccl20, Cxcl1, or Cxcl2 (data not shown when no change is observed). In contrast, DEX did not affect transcription of IL-17/IL-23 and TNF signatures, with the exception of a slight down-regulation of Ccl3/Mip1alpha.

The concentrations of IL-17/IL-23 and TNF signature cytokines (TNF-alpha, IL-1ß, IL-6, LIF, IL-10, and IL-23) and chemokines (CCL2/MCP-1, CCL3/MIP-1alpha, and CCL20/MIP-3alpha) released into the supernatant were compared in the TPH-1 model treated for 24 h with different concentrations of 2,4-D, RPM, and DEX, as described above. As shown in FIG. 16, 2,4--D strongly suppressed the release of IL-17/IL-23 and TNF signature cytokines and chemokines in a dose-dependent manner (>90%; p<0.01). In contrast, and as expected, RPM slightly inhibited the release of IL-1ß, IL-10, TNF-alpha, CCL2/MCP-1, CCL3/MIP-1alpha, and CCL20/MIP-1alpha (<50% at 20 μM; p<0.05) while LIF, IL-6 and IL-23 remained unchanged. DEX strongly suppressed the release of IL-1ß, IL-6, and IL-23 (>90% at 80 ng/ml; p<0.01) and moderately reduced the release of IL-10, CCL2/MCP-1, CCL3/MIP-1alpha, and CCL20/MIP-3alpha (50-90%, p<0.01) while LIF and TNF-alpha remained unchanged.

Thus, we have demonstrated here that treatment with 2,4-D results in immediate transcriptional and translational suppression of IL-17/IL-23 and TNF signaling pathways in vitro. These findings indicate that 2,4-D transcriptionally and translationally suppresses the IL-17/IL-23 and TNF signature cytokines (including IL-1ß, IL-23, LIF, TNF, and COX-2) and chemokines (CCL2, CCL3, and CCL20) but down-regulates only the release of IL-6 and IL-10. Given the difference in modulation of the IL-17/IL-23 and TNF signaling pathways between 2,4-D, RPM, and DEX, these results further indicate that it is 2,4-D, rather than RPM or DEX, which is most effective in switching off the IL-17/IL-23 and TNF pathways.

### Example 10. Effects of 2,4-D on Acute Gouty Arthritis in a Mouse Model

Gout is a painful inflammatory disease caused by the over-production of uric acid, which forms deposits and crystallizes as monosodium urate (MSU) crystals in articular and periarticular tissues, and which trigger an intense inflammatory response and increase the recruitment of neutrophils to the joint.

Currently, management of gout flares relies on the use of NSAIDs, colchicine, corticoids, or biological agents. However, the use of these drugs lack safety in patients with comorbidities, often cause severe side effects (NSAIDs, colchicine, and corticoids), present high cost (biological agents such as IL-1 inhibitor Rilonacept or monoclonal anti-cytokine antibodies), or possess non-satisfactory analgesic effects in some patients. (Rees et al., 2014; Dalbeth et al., 2016)

The effect of 2,4-D in a model of MSU-induced gout in mice was evaluated here.

### Materials & Methods

2,4-D, isolated and purified from Chloranthus spicatus (purity≥98%), was purchased from BOC Science (Shirley, NY). Colchicine (CLC) (purity≥96%) and dexamethasone (DEX) (purity≥98%) were purchased from Sigma. Mice (six per group) were treated with 2,4-D (100 mg/kg or 20 mg/kg, i.p.), CLC (1 mg/kg or 0.2 mg/kg, i.p.), DEX (0.5 mg/kg or 0.1 mg/kg, i.p.), or vehicle (20% polyoxyethylenesorbitan monooleate (Tween 80) plus PBS) 30 min before intra-articular stimulus with MSU (100 μg/10 μL, i.a.). Six mice with intra-articular injection with PBS alone served as a negative control.

Preparation of monosodium urate crystal (MSU). MSU crystals were prepared as described previously (Ruiz-Miyazawa et al., 2017). Briefly, 800 mg of monosodium urate was dissolved in 155 ml of boiling milli-Q water containing 2N NaOH. After pH adjustment to 8.9, the solution was cooled gradually by stirring at room temperature and crystallized overnight at 4° C. Crystals were collected, and centrifuged at 3,000×g for 2 min at 4° C. The crystals were evaporated and sterilized by heating at 180° C. for 2 h and stored in a sterile environment until use. The load of endotoxin present in the MSU crystals was determined using a ToxinSensor^{T}M Single Test Kit (GenScript). The selected dose and concentration of MSU did not present detectable endotoxin levels, as previously described (Ruiz-Miyazawa et al., 2018).

Induction of MSU-Induced Knee Joint Inflammation. Male ICR (CD-1) mice (35-45 g, 10-12 weeks) were purchased from VitalRiver Inc (Beijing, China) and were kept in an animal care facility under controlled temperature of 21±1° C., humidity, and on a light/dark cycle, with ad libitum access to water and standard laboratory chow diet. Animal care and handling procedures were approved by the Animal Ethics Committee of GZUCM (Guangzhou University of Chinese Medicine). All efforts were made to minimize animal suffering and to reduce the number of animals used. Joint inflammation was induced by the intra-articular (i.a.) administration of MSU (200 μg/20 μl) into the right and left knee joints of mice under inhaled 3% isoflurane anesthesia. Control animals received an injection (i.a.) of sterile PBS (20 μL).

Evaluation of Knee Joint Edema. Knee joint edema was determined with a caliper (Mitutoyo) before (zero time), and after the intra-articular injection with MSU at the times indicated. Knee joint edema was determined for each mouse by the difference between the time points indicated on figures and the zero time (Ruiz-Miyazawa et al, 2017). The edema value is represented as Amm/joint.

Histopathological Analysis. Knee joint was collected 20 h after MSU injection, fixed with 10% paraformaldehyde in PBS, and then decalcified for 10 days with EDTA and embedded in paraffin for histological analysis. The paraffin sections were stained with hematoxylin and eosin for conventional morphological evaluation. Results are expressed as leukocytes infiltrate (cell/field) counted at the inflammatory foci within synovial cavity as indicative of synovitis. A dimension used for the analysis of the slices was 1-2 mm² and magnification ×200 by Image J software.

Cytokine Measurement. Mice were anesthetized and sacrificed. Knee joints were collected and frozen in liquid nitrogen. Samples were then homogenized in 500 μl of buffer containing protease inhibitors, centrifuged (3600 rpm, 4° C., 15 min) and supernatants were used to determine Th1/Th2/Th17 cytokine levels using a multiplex microassay (Qantibody® mouse TH17 Array 1 kit, Cat. No.: QAM-TH17-1; RayBiotech, Norcross, GA). Results were expressed as picograms (pg) of cytokine/mg of tissue.

Statistical Analysis. Data were analyzed using GraphPad Prism statistical software (GraphPad Software, Inc., United States-500.288, version 5.0). Results are presented as means±SEM or SD of measurements made on 6 mice per group, with statistical significance determined by two-way ANOVA, comparing each group to the control (“Model”) group. For in vitro experiments, microassays were performed in quadruplicate. p<0.05 was considered significant (*p<0.05, **p<0.01, ***p<0.001).

### Results

### 2,4-D Inhibits MSU-Induced Edema in a Dose-Dependent Manner.

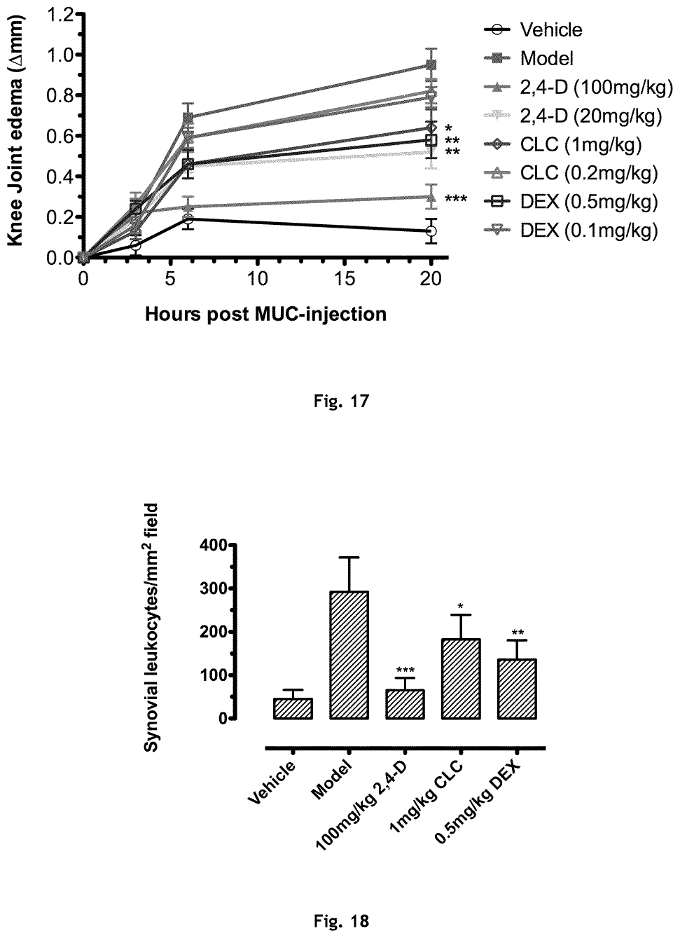

MSU injection induced knee joint edema at all evaluated time-points with a peak (86%, p<0.001) at 20 h (FIG. 17). By 20 h, 2,4-D reduced MSU-induced knee joint edema by 45.3% at dose of 20 mg/kg (p<0.01) and by 68.4% at a dose of 100 mg/kg (p<0.001). In comparison, colchicine (CLC), a drug frequently used to treat acute gout, reduced MSU injection induced knee joint edema by only 32.6% at a high dose (1 mg/kg) (p<0.05). DEX reduced MSU-induced knee joint edema by 16.8% at a dose of 0.1 mg/kg (p<0.05) and by 38.9% 0.5 mg/kg (p<0.01) (FIG. 17).

### 2,4-D Inhibits MSU-Induced Leukocyte Recruitment to the Synovial Cavity of Knee Joint.

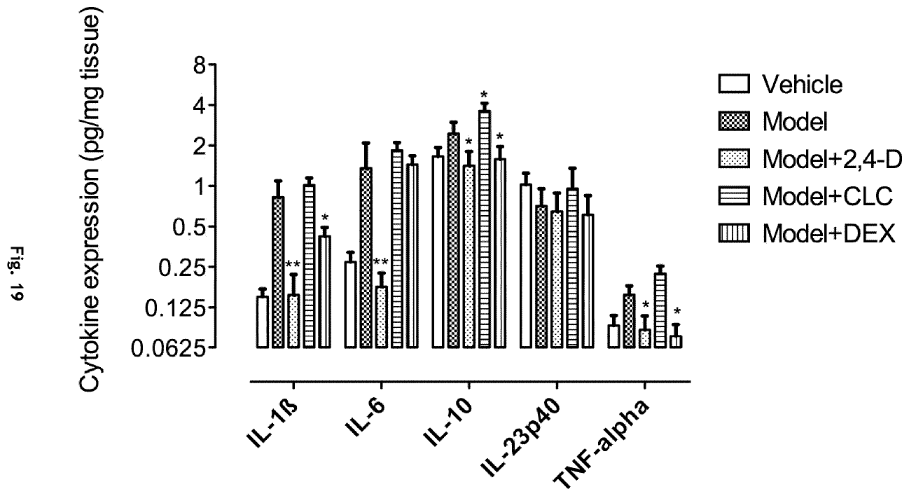

Due to the significant decrease of MSU-induced knee joint edema that was observed at the highest doses of 2,4-D (100 mg/kg), CLC (1 mg/kg), and DEX (0.5 mg/kg), further investigations focused on comparing different high-dosing drug groups. MSU-induced leukocyte recruitment was observed in the synovial cavity of knee joint (data not shown). By 20 h, MSU-induced leukocyte recruitment reached 6.5-fold (p<0.001) (FIG. 18). 2,4-D, at a dose of 100 mg/kg, reduced MSU-induced leukocyte recruitment by 4.5-fold (p<0.001). However, the dose of 1 mg/kg of CLC only marginally inhibited MSU-induced leukocyte recruitment (1.6-fold; p<0.05). DEX, at dose of 0.5 mg/kg, reduced inflammatory cell recruitment to the knee joint by 2.2-fold (p<0.01) (FIG. 18).

### 2,4-D Modulates MSU-Induced Cytokine Production in Knee Joint Tissue.

Among 18 Th1/Th2/Th17 murine cytokines (IL-1ß, IL-2, IL-4, IL-5, IL-6, IL-10, IL-12p70, IL-13, IL-17, IL-17F, IL-21, IL-22, IL-23, IL-28, INF-gamma, MIP-3a, TGF-beta, and TNF-alpha), 7 pro-inflammatory cytokines (IL-1ß, IL-6, IL-10, IL-12p70, IL-23, INF-gamma, and TNF-alpha) were detected in mice knee joint samples. Among these 7 cytokines, only 4 (IL-1ß, IL-6, IL-10, and TNF-alpha) were up-regulated by MSU treatment. 2,4-D decreased IL-1ß, IL-6, IL-10, and TNF-alpha cytokines in knee joint tissue by 5.3- (p<0.01), 7.6- (p<0.01), 1.7- (p<0.05), and 1.9-fold (p<0.05), respectively (FIG. 19). CLC slightly increased (1.5-fold, p<0.05) the expression of IL-10 and had no effect on other pro-inflammatory cytokines. DEX reduced IL-1ß, IL-10, and TNF-alpha cytokines in knee joint tissue by 1.9- (p<0.05), 1.5- (p<0.05), and 2.0-fold (p<0.05), respectively, but had no effect on IL-6.

2,4-D is effective in suppressing MSU-induced leukocyte recruitment and secretion of pro-inflammatory cytokines in the knee joint (synovial) tissue in the MSU-induced gout arthritis mouse model. 2,4-D notably inhibited MSU-induced leukocyte recruitment to the synovial cavity to a greater extent than either CLC or DEX.

### Example 11. 2,4-D Improves the Phenotype of a Mouse Model of Amyotrophic Lateral Sclerosis (ALS)

Amyotrophic lateral Sclerosis (ALS) is a neurodegenerative disease that results in the progressive loss of motor neurons that control voluntary muscles. ALS is the most common type of motor neuron disease. Early symptoms of ALS include stiff muscles, muscle twitches, and gradual increasing weakness and muscle wasting. Motor neuron loss continues until the ability to eat, speak, move, and finally the ability to breathe is lost. There is no specific treatment for ALS and approximately half of the patients die within the 3 first years of their symptoms beginning (Fernández-Ruiz, et al., 2021, Br J Pharmacol 178, 1253-1256. Most cases of ALS (about 90% to 95%) have no known cause, and are known as sporadic ALS. Both genetic and environmental factors are however believed to be involved. The remaining cases, known as familial ALS are likely of genetic origin, the specifically involved genes having being identified (such as SOD1, see below) in about half of these cases. Although probably not the primary cause of neurodegeneration in ALS patients, neuro-inflammation is a common feature in ALS and participates in the acceleration of neuronal death (McCombe P. A. et al., 2020, Front Neurol 11, 279). Recent studies have demonstrated that during disease progression, the phenotype of peripheral helper T cells shifts from Treg cells to pro-inflammatory Th1 and Th17 cells, which become more abundant with increasing clinical severity (Jin, R. et al., 2020, Scientific reports 5941, 5941).

The effect of 2,4-D in a mouse model of Amyotrophic lateral Sclerosis (ALS) was evaluated here.

### Materials & Methods

Animals. The ALS mouse model is the SOD1 G93A (Gurney et al. Science 1994). Mutations in the SOD1 gene represent about 20% of the mutations in human familiar forms of ALS. Mice with multiple human transgenes with the G93A mutation in the SOD1 gene develop a progressive muscle paralysis and a loss of spinal motor neurons as in human patients. We used the high expressor lines (Jackson Labs #002726 B6SJL-Tg(SOD1*G93A)1 Gur/J) that contains at least 15 copies of the human transgene. SOD1 G93A males are crossed with B6/SJL females (Janvier Labs #SH-B6SJL-F1; hybrid model B6SJLF1/JRj). Genotyping allows us to identify the transgenic animals in the litter.

Symptoms progression is assessed using a neuroscore grid as in Hatzipetros et al. JoVE 2015. Mice are euthanized when they are transitioning from neuroscore 2 to neuroscore 3 (full paralysis of one hindlimb). This transition happens about 120-130 days post-natal in our breeding conditions and in the absence of the 2,4-D treatment. Non transgenic littermates are used as control animals.

Chronic administration of 2,4-D in mice. Low solubility of 2,4-D in water prevents us to administrate the compound in drinking water. Daily delivery of the drug for a long time period (about 100 days) is not compatible with IP injections for ethics considerations and also because of the infection risk. We then rely on oral administration in a gel using a method modified from Lei Zhang, Star Protocol 2021. Briefly, the gel is produced by mixing alimentary gelatin (8%, Vahiné) in a 2% sucralose solution (Myprotein, zero calory, no flavor) and by heating this mixture at 60° C. for 20-30 minutes until clarification. An artificial flavor, for instance “cheese flavor” (Arôme Alimentaire Emmental 58 ml Cuisineaddict), 2 microliters/milliliter), may be added to this preparation along with some kibble grinds to powder (the same kibble as the one used in the house facility for ordinary diet) for improving palatability. This mixture is then aliquoted in individual daily portions of 315 microliters. 1 to 2 mg of 2,4-D (depending on the mouse body weight, 50 mg of 2,4-D per kg of mouse body weight) is added to each portion while the mixture is still warm (2,4-D is not added in the vehicle portions given to untreated animals). This mixture solidifies in a jelly form upon cooling. The gel can be kept in a fridge (4° C.) for 6 days. We usually have 4 mice (males or females) per cage. To feed the mice with 2,4-D we remove the ordinary kibbles (sometime 4 hours before) and we physically isolated each mouse in a corner of the cage with a plexiglass cross placed in the cage to separate four compartments. One portion jelly is placed at the bottom of each compartment until the mouse has eaten it. In the case the mouse has not eaten the full portion after 3 hours, we evaluate how much was taken by the mouse. The mouse feeding with 2,4-D jelly can be started after weaning (21 days in our animal house). In the case we wish to expose the mice with 2,4-D earlier, we give the jelly to the pregnant mother or to the nursing mother.

### Results

### Effects of 2,4-D on the Progression of Motor Symptoms

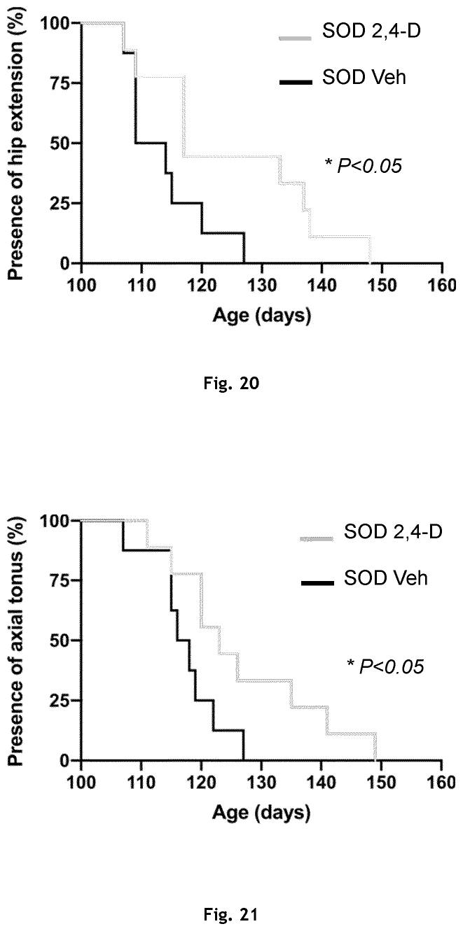

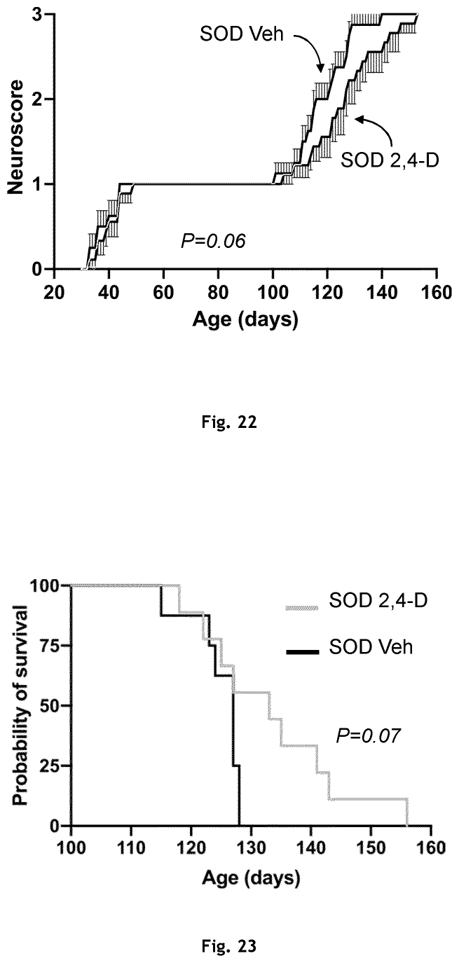

A daily feeding of SOD1 G93A mice with 2,4-D starting 21 days post-natal until reaching neuroscore 3 (at which there are euthanatized) significantly extends the times at which the full hindlimb extension (FIG. 20) and the axial tonus (FIG. 21) are loss compared with the mice that received the vehicle only (gel without 2,4-D). In addition, the neuroscore progression (from 0 to 3) tends to slow down in the treated mice (FIG. 22), and the time at which neuroscore 3 is reached (survival time; FIG. 23) tends to be delayed in the same mice. These results indicate that 2,4-D slows down the disease progression of SOD1 G93A mice.

### Example 12. 2,4-D Reduces the Grading Score in a Rat Model of Human Wet Macular Degeneration (AMD)

Macular degeneration, also known as age-related macular degeneration (AMD) is a medical condition, which eventually results in blurred or no vision in the center of the visual field. While it does not result in complete blindness, loss of central vision markedly impairs the activities of daily life. Macular degeneration, which typically occurs in older people is due to damage to the macula of the retina and divided into “dry” and “wet” forms. In wet form of AMD, blood vessels grow under the macula, causing blood and fluid to leak into the retina (Ambati J. et al., 2012, Neuron July 12; 75:26-39). In wet AMD, anti-VEGF medication injected into the eye or less commonly laser coagulation or photodynamic therapy may slow worsening. Many studies have also shown the key role inflammation plays in the pathogenesis of wet ARMD (Hadziahmetovic M. et al., 2021, Frontiers in Cell and Developmental Biology, 8:612812).

The effect of 2,4-D in a rat wet macular degeneration model was evaluated here.

### Materials & Methods

Animals. All experiments were performed in accordance with the European Communities Council Directive 86/609/EEC and approved by local ethical committees (#2541-2015110210279792 v3). Six- to eight-week-old male Long Evans rats from the Janvier Breeding Center (Le Genest-Saint-Isle, France) were used to create a rat model of choroidal neovascularization (CNV) used as a model of human wet macular degeneration.

Animals were kept in pathogen-free conditions with food, water and litter and housed in a 12-hour light/12-hour dark cycle. Anesthesia was induced by intramuscular ketamine 40 mg/kg and xylazine 4 mg/kg. Animals were sacrificed by carbon dioxide inhalation or cervical dislocation

Laser-induced CNV. After anesthesia and dilation of the pupils, coverslips were positioned on the cornea as a contact glass. For rats, six or eight burns were performed 2 to 3 optic disc diameters away from the optic nerve with an Argon laser (532 nm) mounted on a slit lamp (175 mW, 0.1 s and 50 μm). Both eyes of animals received laser induction. The presence of a bubble witnessed the rupture of Bruch's membrane and confirmed a successful laser impact.

Treatments. After laser photocoagulation, rats were divided into 3 treatment groups: —Intravitreous injection (IVT) of 2,4-D (identified as “new treatment”, New T), —IVT of vehicle (PBS), —IVT of triamcinolone acetonide (Kenacort® 40 mg/ml; TA) (n=12 rats per group). The IVT consisted of 5 μl injected with a 31G needle mounted on a micro-thin (100 μl) syringe posterior to the limbus, avoiding the lens. IVT was performed immediately after laser had been performed. Animals were sacrificed at 7 days (n=6 per group- and at 16 days (n=6 per group).

Fluorescein angiography (FA). FA was performed 14 days after laser induction. After pupil dilatation, fluorescein (0.2 mL of 10% fluorescein in saline) was injected intravenously in the tail. Early and late phase angiograms were recorded 1-3 and 5-7 min respectively after fluorescein injection. Simultaneously, infrared images were acquired to detect the site and effective presence of laser burn. For each laser-induced lesion, fluorescein leakage was graded qualitatively by evaluating the increase in size/intensity of dye between the early and late phases. Angiographic scores were established by 2 blinded observers according to the following criteria: grade 0, no hyper-fluorescence; grade 1, slight hyper-fluorescence with no increase in intensity nor in size; grade 2, hyper-fluorescence increasing in intensity but not in size; grade 3, hyper-fluorescence increasing both in intensity and size; grade 4, hyper-fluorescence size increase more than 2-diameter of the initial laser burn.

RPE-choroid flat-mounts and CNV quantifications. Two days after FA examination (time necessary for fluorescein elimination), eyes were enucleated, fixed in 4% PFA for 15 min at room temperature and sectioned at the limbus; the cornea and lens were discarded. The retina was separated from the RPE-choroid complex. Eight radial incisions were made on the RPE-choroid, which was then flat-mounted and post-fixed with acetone for 15 min at −20° C. After washing with 0.1% Triton x100 in PBS, FITC-GSL I-Isolectin B4 (1:200, Vector, AbCys, Paris, France) was applied over night at −4° C. After washing with PBS, the RPE-choroid was flat-mounted and observed with a confocal microscope (Zeiss LSM710, Le Pecq, France). Images of the CNV were captured with a digital video camera coupled to a computer system on an Olympus fluorescence microscope. Bruch ruptures could be easily observed at each laser spot. The area of CNV-related fluorescence on each horizontal section was measured using the ImageJ software.

Immunofluorescence of RPE-choroid flat-mounts. Three days after laser induction (peak of macrophage infiltration), RPE-choroid flat-mounts were also prepared for immunofluorescence. A polyclonal rabbit anti-IBA1 antibody (1:400, Wako, Neuss, Germany) was applied over night at −4° C. After washing with 0.1% Triton X100/PBS, the flat-mounts were incubated with AlexaFluo® 594 goat anti-rabbit IgG (1:200, Molecular Probes, Leiden, The Netherlands), the nuclei were counterstained with DAPI. Images of IBA1 positive macrophage/microglia were observed and captured with a fluorescent microscope.

### Results

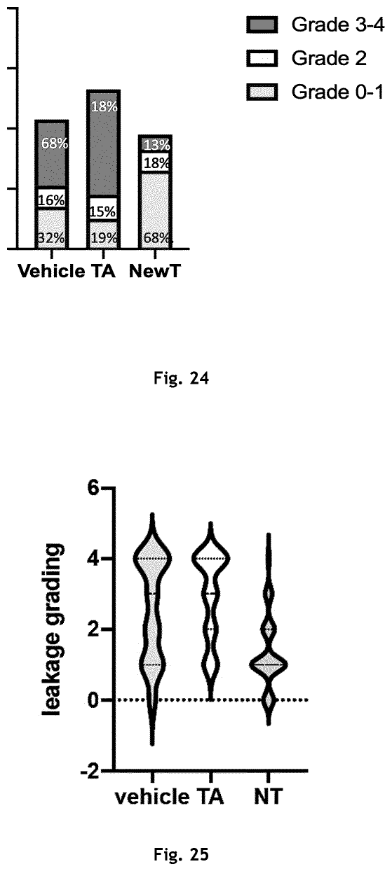

At 14 days, fluorescein angiography grading showed a reduction in the percentage of impact with intense leakage (grade 3 and 4) in the 2,4-D (NewT) group as compared to vehicle and TA treated groups (FIG. 24), with 68% of impact in grade 3-4 in the vehicle group as compared to 13% in the 2,4-D (NewT) group. There was a doubling of percentage of impact graded 0-1 in the 2,4-D (NewT) group as compared to vehicle (68% as compared to 32%). Statistical analysis showed that 2,4-D (New T) significantly reduced the grading score of impacts as compared to both vehicle and TA (FIG. 25). Dunn's multiple comparison tests between groups: vehicle vs TA: ns; Vehicle vs NT: p=0.0003, TA vs NT: p<0.0001.

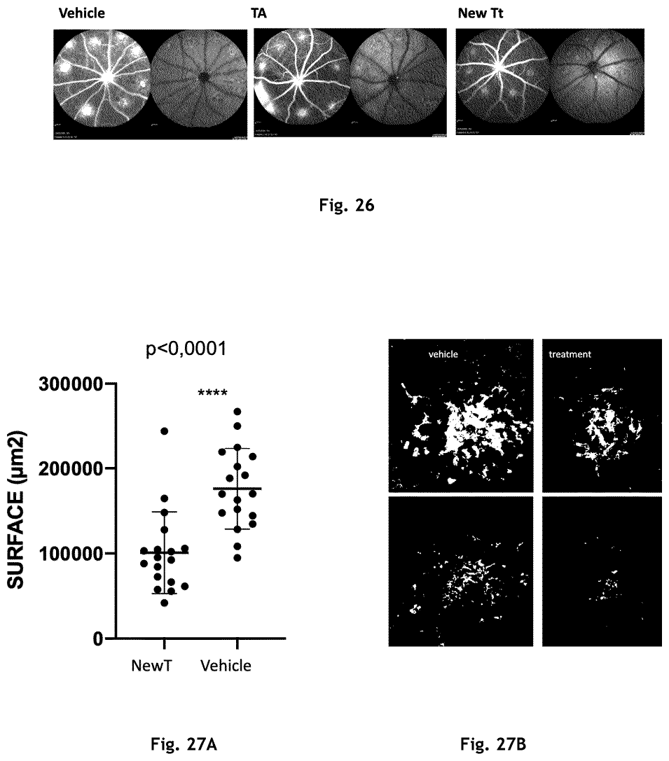

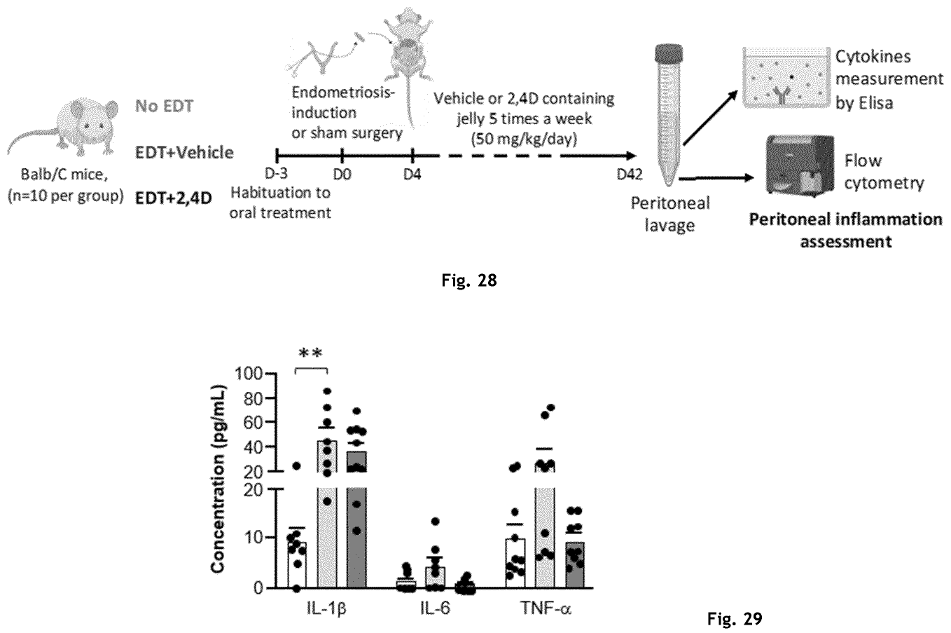

Examples of FA images and infrared images for treated eyes are shown in FIG. 26. The surface of CNV, analyzed by measuring the lectin positive labelling of flat Mounted CNV, showed that 2,4-D (NewT) significantly reduced the CNV size. In addition, an important reduction in the number of infiltrating IBA-1 positive macrophages was observed in CNV of the 2,4-D- (NewT) treated eyes.

### Example 13. 2,4D Treatment Limits the Peritoneal Inflammation Induced by Endometriosis (EDT)

Endometriosis is a disease of the female reproductive system in which cells similar to those in the endometrium, the layer of tissue that normally covers the inside of the uterus, grow outside the uterus, mostly in tissues around the uterus and ovaries. This condition causes pain and infertility. Pain can be chronic, occurring during menstruation and can be exacerbated during sexual intercourse. There is no cure for endometriosis, but some treatments may improve symptoms: pain medications, hormonal treatments (such as birth control pill) or surgery to remove the ectopic endometrial tissue. The recommended pain medication is usually a non-steroidal anti-inflammatory drug. Endometriosis is indeed associated with inflammation, characterised by altered immunological functions and enhanced numbers of activated peritoneal immune cells. (Lousse J C et al., 2012, Front Biosci; 4:23-40). It has been proposed that alteration/imbalance of regulatory T cells and Th17 cells play an important role in the pathophysiology of endometriosis (Khan K N et al., 2019, J Clin Endocrinol Metab, 104:4715-4729). Because of its control on Th17 maintenance and proliferation, inhibition of IL-23 might ameliorate endometriosis-associated inflammation and consequent pain in patients.

The effect of 2,4-D in an induced-endometriosis mouse model was evaluated here.

### Materials & Methods

Experimental design. Balb/C female mice (10 weeks old) were habituated for 3 days to take a jelly containing (group EDT+2,4D) or not (groups No EDT and EDT+Vehicle) the compound. Surgical implantation of endometrial tissue from syngenic female in peritoneum, was performed at day 0 for groups EDT+Vehicle and EDT+2,4D. No EDT group had a sham surgery. The vehicle or treatment was then given 5 times a week (50 mg/kg/day) during 6 weeks. Mice were then sacrificed and the peritoneal inflammation was assessed. All the details of the protocol are depicted in FIG. 27.
Cytokine measurement in cell-free peritoneal lavage. After mice were euthanized, 5 mL of PBS was injected into the peritoneal cavity and the diluted peritoneal fluid containing the cells was then retrieved. The cells were pelleted by centrifugation at 500×g 4° C. and the supernatant (cell-free peritoneal fluid) was frozen at −80° C. for subsequent cytokine concentration measurement by ELISA. Cytokine concentration was assessed by ELISA kit in the peritoneal fluid (ThermoFisher, Mouse IL-1β, IL6 and TNFalpha Uncoated ELISA). To determine the concentrations of cytokines, these kits used specific primary and secondary antibodies, revealed by the HRP-TMB color reaction. The optical density was read with the Clariostar Plus Microplate Reader (BMG LABTECH), through the Clario software. Concentrations were calculated from a standard curve according to the manufacturer's protocol.
Phenotypic study of immune cells from the peritoneal cavity assessed by flow cytometry. The pelleted peritoneal cells were resuspended in PBS-2% FBS and counted. One million cells were stained with the following fluorophore-conjugated antibodies purchased from BioLegend: CD45 BV510, CD11b APC-Cy7, CD11c PE, F4/80 BV711, CD80 PE Cy5, CD206 AF647, Ly-6C, PE-Cy7, CCR2 BV786 et CXCR4 BV421, CD45R/B220 PE Texas-Red, CD3E FITC, NK1.1 BV605 and CD69 PerCP-Cy5.5, Zombie-UV. Cells were incubated 20 min in the dark at 4° C., washed with PBS-2% FBS, and were fixed with paraformaldehyde 4% in 5 mL round-bottom polystyrene tubes (Falcon™ 352054). Compensation was adjusted with Ultracomp beads (ThermoFisher). All the antibodies were diluted at 1/200. All data were collected on LSR Fortessa flow cytometer (BD Bioscience, USA) and analyzed with FlowJo software.
Statistical analysis. Outlier values farther than 2 standard deviation from the mean were not taken into account. Kruskal Wallis non-parametric test followed by Dunn's multiple comparisons test were performed for cytokines, One-Way Anova with Tukey's multiple comparisons test for flow cytometry data. *p<0.05, **p<0.01, ***p<0.001.

### Results

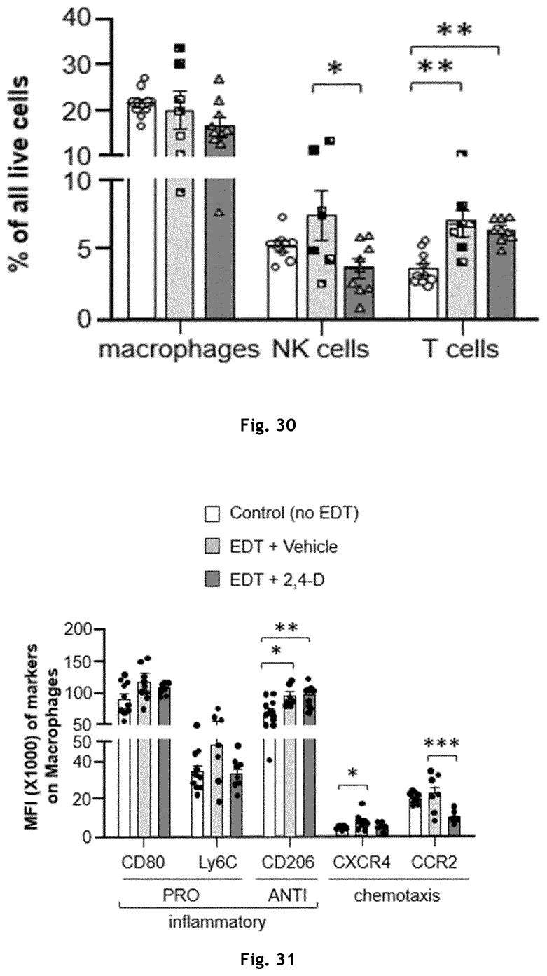

Endometriosis led to a significant increase of the pro-inflammatory cytokine IL1-β and a tendency towards an increase of two other inflammatory cytokines (IL-6 and TNF-α). 2,4D tended to normalize the concentration of these cytokines, as shown in FIG. 29. endometriosis tended to increase the proportion of NK cells amongst the peritoneal cells and increased the T cell proportion. Study of the phenotype of the peritoneal cells shows that the treatment with 2,4D significantly reduced the proportion of NK cells, but did not affect global T cell proportion. Proportion of macrophages, the most abundant cell type, was not changed by endometriosis or 2,4D (FIG. 30). However cell surface macrophage markers were altered: endometriosis tended to increase the pro-inflammatory markers (CD80 and Ly6C) and significantly increased the anti-inflammatory marker CD206. As shown in FIG. 31, 2,4D treatment tended to decrease the Ly6C pro-inflammatory marker, but did not reduce the anti-inflammatory CD206 marker. Concerning surface molecule important for chemotaxis, two chemokine receptors (CXCR4 and CCR2) were assessed. Endometriosis increased CXCR4, and tended to increase CCR2. 2,4D treatment tended to decrease CXCR4 and significantly reduced CCR2.

Taken all together, these results strongly support a limitation of endometriosis-induced inflammation by 2,4D treatment.

### CONCLUSIONS

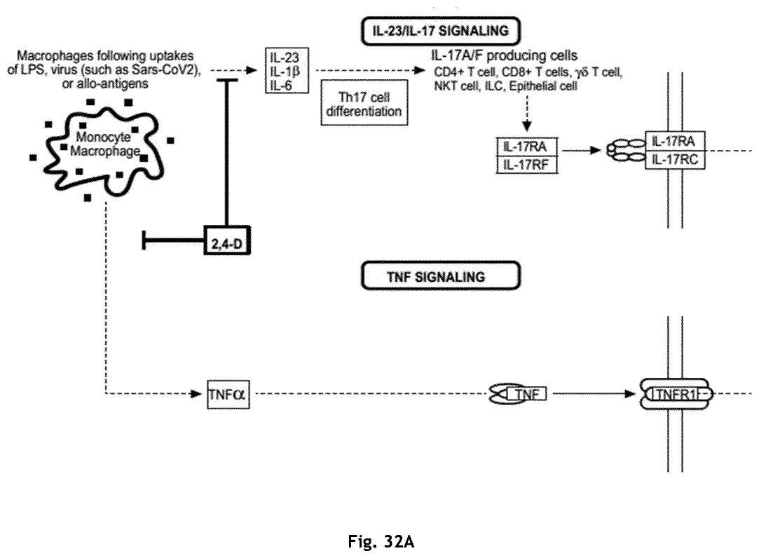

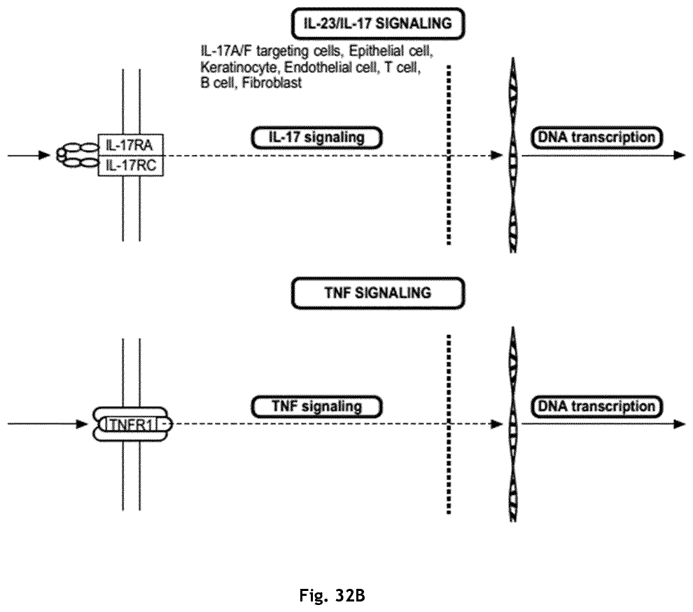

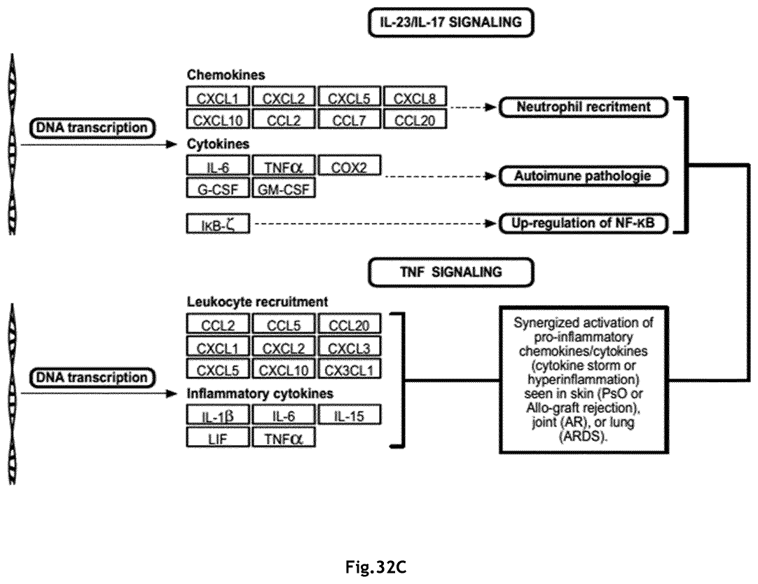

As illustrated herein, a composition comprising 2,4-D not only strongly inhibits IL-23/IL-17 axis-related gene expressions in a human monocyte-derived cell line expressing the IL-23 gene, the administration of 2,4-D in seven animal models, each reproducing a disease associated with IL-23/IL-17 axis activation, successfully alleviated symptoms observed with said diseases. Indeed, 2,4-D was able to successfully treat diseases ranging from psoriasis, rheumatoid arthritis, acute gout arthritis, and graft rejection to sclerosis lateral amyotrophic, macular degeneration and endometriosis in the corresponding mouse models. 2,4-D specifically inhibited IL-23/IL-17 axis, a key cytokine signaling pathway associated with each of these diseases, in all of these models. These findings clearly demonstrate that 2,4-D targets the IL-23/IL-17 axis and is beneficial for treating patients with both acute and chronic autoimmune diseases, as well as superinflammatory or degenerative diseases. Without being bound by theory, a mechanism of action of 2,4-D is shown in FIG. 32. Of note, the (E) isomer surprisingly has 5-fold higher activity than the (Z) isomer.

### REFERENCES

Aggarwal et al., 2003, J Biol Chem, 278(3): 1910-4.
Aggeletopoulou et al., 2018, World J Gastroenterol., 24(36): 4093-4103.
Alsheikh et al., 2019, The Egyptian Rheumatologist, 41(2): 99-103.
Alyasin et al., 2017 Iran. J. Immunol., 14(1): 73-80.
Ambati J et al., 2012, Neuron July 12; 75:26-39
Auyeung et al., 2005. The Journal of infection, 51(2), 98-102.
Bai et al., 2014, Int J Mol Med. 33(3): 711-21.
British Association of Dermatologists, “Guselkumab,” March 2019.
Chanput et al., 2010, Food & function. 1(3): 254-61.
Chanput et al., 2014, Int Immunopharmacol. 23(1): 37-45.
Cheng et al, 2017, J Vis Exp. (119), e55105.
Cron and Chatham. 2020, J Rheumatol. 47(5):639-642.
Dalbeth et al., 2016, Gout. Lancet, 388, 2039-2052.
Das et al., 2009, Blood. 113(10): 2352-62.
Debnath and Berk, 2017, Mol Neurobiol. 54(10):8170-8178.
Dedong et al., 2019, Immunol Lett. 210: 33-39.
Di Meglio and Nestle, 2010, F1000 Biol Rep. 2: 40.
Fernández-Ruiz J. et al., 2021, Br J Pharmacol 178, 1253-1256
Fiala et al., 2010, J Neuroinflammation. 7: 76.
Fragoulis et al., 2016, Annu Rev Med. 2016; 67:337-53.
Foulkes et al., 2019. Drugs in Context; 8: 1-11.
Gurney M E, et al. 1994 Science 264(5166):1772-5; Erratum 1995 Science 269(5221):149.
Hadziahmetovic M. et al., 2021, Frontiers in Cell and Developmental Biology, 8:612812
Haugh et al., 2018. Drug Design, Development and Therapy; 12: 3879-3883.
Happel et al., 2005, J Exp Med, 202(6): 761-769.
Hatzipetros T, et al., 2015, J Vis Exp. 104:53257.
Hernández-Santos and Gaffen, 2012, Cell Host Microbe; 11(5): 425-435.
Hu et al., 2021. J Med Virol. 93(1): 250-256.
Imai et al., 2005, Nature, 436(7047): 112-116.
Jiang et al. 2018, Oncotarget, 9(47): 28309-28321.
Jin, R. et al., 2020, Scientific reports 5941, 5941
Khan K N et al., 2019, J Clin Endocrinol Metab, 104:4715-4729
Kolli et al., 2019, Ann Pharmacother, 53(4):413-418.
Lee et al., 2017, Am J Physiol Lung Cell Mol Physiol. 312: L13-L21.
Li, et al., 2011, Neuroimmunology, 234(1-2): 155-160.
Li et al., 2015. J Immunol, 195(1), 87-95.
Li and Zhou, 2019, Tohoku J. Exp. Med., 247: 87-98
Liu, et al., 2012, Atherosclerosis, 221(1): 232-241.
Lousse J C, et al., 2012, Front Biosci 4:23-40
McCombe P. A. et al., 2020, Front Neurol 11, 279
Meeks et al., 2009, J Immunol, 183: 8026-8034.
Mensah-Brown, 2006, J Immunol. 36(1):216-23.
Murphy et al, 2003, J. Exp. Med. 198:1951-1957.
Nogueira, et al., 2010, Nephrol Dial Transplant. 25(7):2209-17
Okamato et al. 2015, J Immunol. 194(3): 1357-63.
Paust et al., 2009, J Am Soc Nephrol. 20(5): 969-979.
Pepple and Lin, 2018, Ophthalmology. 125(12):1977-1983.
Puig, 2019, Expert Rev Clin Immunol,15(6):589-597.
RECOVERY Collaborative Group, et al. (2020) Dexamethasone in Hospitalized Patients with Covid-19—Preliminary Report [published online ahead of print, 2020 Jul. 17]. N Eng/J Med. NEJMoa2021436.
Rees et al., 2014, Nat. Rev. Rheumatol. 10, 271-283.
Remington: The Science and Practice of Pharmacy, 22nd edition, Loyd V. Allen, Jr, editor. Philadelphia, PA: Pharmaceutical Press. 2012.
Roman et al., Ther Clin Risk Manag. 11: 1767-1777.
Ruiz-Miyazawa et al., 2017, Inflammopharmacology, 25, 555-570.
Ruiz-Miyazawa et al., 2018, J. Agric. Food Chem. 66 6269-6280.
Ryba-Stanisfawowska et al., 2013, Human Immunology, 74(6): 701-707.
Tang et al., 2012, Immunology. 135(2): 112-124.
Uygun et al., 2019, J Child Neurol. 34(13):815-819.
Villegas et al., 2019, J Autoimmun. 98:59-73.
Wang et al., 2018, ACG Case Rep J; 56:1-3.
Wang et al., 2013, PLoS Pathog. 9(6):e1003410.
Wong et al., 2008, Clinical Immunology, 12(3): 385-393.
Xueyi et al., 2013, Journal of Clinical Immunology, 33(1): 151-161.
Yang et al., 2019, Drug Design, Development and Therapy, 13:1993-2000.
Ye et al., 2015, PLoS One. 10(1):e0117704.
Ye et al., 2020, The Journal of infection, 80(6), 607-613.
Zheng et al., 2018, Front Immunol. 9: 96.
Zihni Bilik et al., 2016, Medicine (Baltimore). 95(18): e3562.
Zhang et al., 2013, PLoS ONE, 8(12): e71466.
Zhang L. 2021, STAR Protoc. 2(1):100330.
Zizzo et al., 2011, Clinical Immunology, 138(1):107-116.

1. A method for preventing and/or treating a disease associated with IL-23 expression, except cancers, in a subject in need thereof, comprising administering to the subject an effective amount of a composition comprising 2′,4′-dihydroxy-3′,6′-dimethoxychalcone or a pharmaceutically acceptable salt thereof, wherein 2′,4′-dihydroxy-3′,6′-dimethoxychalcone down-regulates gene expression of IL-23.

2. The method according to claim 1, wherein the composition further comprises a pharmaceutically acceptable carrier.

3. The method according to claim 1, wherein the composition further comprises at least one additional therapeutic agent.

4. The method according to claim 1, wherein 2′,4′-dihydroxy-3′,6′-dimethoxychalcone or the pharmaceutically acceptable salt thereof is the sole therapeutic agent in the composition.

5. The method according to claim 1, wherein the composition is administered via a local enteral, or parenteral route.

6. The method according to claim 1, wherein the composition is administered at least once per day.

7. The method according to claim 1, wherein 2′,4′-dihydroxy-3′,6′-dimethoxychalcone or the pharmaceutically acceptable salt thereof is administered at a dose comprised of 1 mg/kg to 10 mg/kg per day.

8. The method according to claim 1, wherein the disease associated with IL-23 expression is selected from the group consisting of an autoimmune disease, an inflammatory disease including non-infectious inflammatory respiratory diseases, and a neurodegenerative disease other than Alzheimer's.

9. The method according to claim 8, wherein the autoimmune disease is selected from the group consisting of rheumatologic autoimmune diseases, gastrointestinal and liver autoimmune diseases, vasculitis, renal autoimmune diseases, dermatological autoimmune diseases, hematologic autoimmune diseases, atherosclerosis, uveitis, autoimmune ear diseases, Raynaud's syndrome, diseases associated with organ transplantation, and autoimmune endocrine diseases such as diabetes.

10. The method according to claim 8, wherein:
the inflammatory disease is selected from the group consisting of arthritis, rheumatoid arthritis, acute gout arthritis, osteoarthritis, an inflammatory bone disease; an inflammatory lung disease, preferably asthma, adult respiratory distress syndrome, or chronic obstructive airway disease; Behcet's disease; an inflammatory disease of the eye preferably corneal dystrophy, trachoma, onchocerciasis, uveitis, sympathetic ophthalmitis or endophthalmitis; a chronic inflammatory disease of the gums preferably gingivitis or periodontitis; tuberculosis; leprosy; an inflammatory disease of the kidney preferably a uremic complication, glomerulonephritis or nephrosis; an inflammatory disorder of the skin, preferably psoriasis; a chronic demyelinating diseases of the nervous system; infectious meningitis; encephalomyelitis; Parkinson's disease; Huntington's disease; amyotrophic lateral sclerosis; an immune-complex vasculitis; systemic lupus erythematosus (SLE); an inflammatory disease of the heart, preferably cardiomyopathy, coronary thrombosis, ischemic heart disease hypercholesterolemia, or atherosclerosis; preeclampsia; schizophrenia; chronic liver failure, or brain or spinal cord trauma; endometriosis and/or
the neurodegenerative disease is selected from the group consisting of migraine headache, trigeminal neuralgia, myasthenia gravis, a demyelinating disease, preferably acute transverse myelitis; an extrapyramidal and cerebellar disorder; a hyperkinetic movement disorder, preferably Huntington's Chorea or senile chorea; a drug-induced movement disorder, preferably a disorder induced by drugs which block CNS dopamine receptors; a hypokinetic movement disorder, preferably Parkinson's disease; progressive supranucleo Palsy; a structural lesion of the cerebellum; a spinocerebellar degenerations, preferably spinal ataxia, Friedreich's ataxia, a cerebellar cortical degeneration, or a multiple systems degeneration; a systemic disorder, preferably Refsum's disease, abetalipoproteinemia, ataxia, telangiectasia, or mitochondrial multi-system disorder; a disorder of the motor unit, preferably a neurogenic muscular atrophy and more preferably amyotrophic lateral sclerosis; Down's Syndrome in middle age; Diffuse Lewy body disease; Senile Dementia of Lewy body type; Wernicke-Korsakoff syndrome; chronic alcoholism; Creutzfeldt-Jakob disease; Subacute sclerosing panencephalitis, Rasmussen's encephalitis, Huntington's disease, Hallerrorden-Spatz disease; Dementia pugilistica; neurotraumatic injury; inflammatory pain; autism; depression; head trauma, stroke; cognitive disorders; epilepsy; macular degeneration; and/or
the non-infectious inflammatory respiratory disease is selected from the group consisting of interstitial lung disease, cystic fibrosis, pulmonary oedema, chronic obstructive pulmonary disease, asthma, allergic alveolitis, restrictive lung disease, fibrosis, lupus erythematosus, systemic scleroderma or sarcoidosis, and rhinitis.

11. The method according to claim 1, wherein the disease associated with IL-23 expression is selected from the group consisting of graft-versus-host disease (GvHD), psoriasis, acute gout arthritis, rheumatoid arthritis, amyotrophic lateral sclerosis, macular degeneration, and endometriosis.

12. The method according to claim 5, wherein the composition is administered via a topical, oral, intraperitoneal, intravenous, intradermal, subcutaneous, intra-articular, or mucosal route.
